(19) United States
(12) Patent Application Publication
(10) Pub. No.: US 2026/0207076 A1
(43) Pub. Date: Jul. 23, 2026

(54) SYSTEMS AND METHODS FOR MONITORING OF INCENTIVE SPIROMETRY

(71) Applicant: TIDAL MEDICAL TECHNOLOGIES LLC., Plano, TX (US)

(72) Inventors: FARID FARAHMAND, Plano, TX (US); Foad Farahmand, Plano, TX (US)

(21) Appl. No.: 19/568,644

(22) Filed: Mar. 16, 2026

Related U.S. Application Data
(63) Continuation-in-part of application No. 18/495,507, filed on Oct. 26, 2023.
Application No. 18/495,507 is a division of application No. 16/782,840, filed on Feb. 5, 2020, now Pat. No. 11,832,930.
Application No. 16/782,840 is a continuation of application No. 16/174,903, filed on Oct. 30, 2018, now Pat. No. 10,610,129.

(51) Int. Cl. A61B 5/091; A61B 5/00; A61B 5/08

(52) U.S. Cl. CPC A61B 5/091; A61B 5/0803; A61B 5/486; A61B 5/7425; A61B 5/7475

(57) ABSTRACT

An incentive spirometry monitoring device and method of use. The device can be integrated to/made a part of an incentive spirometer and is capable of monitoring inspiration within the incentive spirometer. The incentive spirometry monitoring device has a user interface for inputting a variety of different parameters, including a desired air volume, as well as attempt thresholds. Results can be stored such that medical personnel can review attempts by a patient to monitor therapeutic use, as well as encouraging patient use.

### REFERENCE TO RELATED APPLICATION

The present application is a continuation-in-part of and claims priority to U.S. patent application Ser. No. 18/495,507 filed Oct. 26, 2023 and titled “SYSTEMS AND METHODS FOR MONITORING OF INCENTIVE SPIROMETRY”, which is a divisional of and claims priority to U.S. patent application Ser. No. 16/782,840 filed Feb. 5, 2020 and titled “SYSTEMS AND METHODS FOR PORTABLE MONITORING OF INCENTIVE SPIROMETRY”, which is a continuation of and claims priority to U.S. patent application Ser. No. 16/174,903, filed Oct. 30, 2018 and titled “SYSTEMS AND METHODS FOR PORTABLE MONITORING OF INCENTIVE SPIROMETRY,” each of which is incorporated herein by reference in their entirety.

### TECHNICAL FIELD

The present disclosure relates generally to systems and methods to monitor incentive spirometry. More particularly, the technology is directed to a portable monitoring device configured to monitor use of an incentive spirometer and providing local feedback.

### DESCRIPTION OF THE RELATED ART

Postoperative pulmonary complications, including atelectasis, pneumonia, and respiratory failure, commonly arise in patients following major cardiac, thoracic, and abdominal surgeries. Deep breathing exercises help reduce postoperative pulmonary complications by improving postoperative lung expansion and ventilation. Incentive spirometry, designed to mimic natural yawning or sighing, is routinely prescribed by clinicians as a therapeutic strategy to encourage deep breathing. Incentive spirometry forces the patient to take long, deep breaths, which decreases plural pressure and increases lung expansion and gas exchange. Incentive spirometry is accomplished through use of an incentive spirometer, a device that provides feedback when a patient inhales at a predetermined volume for a minimum of five seconds. Inhalation results in the raising of a piston within the device, and a successful attempt is achieved when the piston raises to a set target volume.

### BRIEF SUMMARY OF THE DISCLOSURE

Embodiments described herein are directed to systems and methods for monitoring incentive spirometry through a portable device.

In some embodiments, an incentive spirometry device comprises a user interface through which a desired air volume is set, the desired air volume comprising a patient-specific incentive spirometry goal. The incentive spirometry device further comprises a sensor measuring inspiratory/expiratory metrics in the incentive spirometry device by tracking displacement of a piston within the incentive spirometer. Further still, the incentive spirometry device comprises a processor configured to determine compliance by a patient inspiring air using the incentive spirometer device based on a summation of counted successful inspiring air events over a period of time, and a display presenting a visual indication indicating compliance or non-compliance relative to the patient-specific incentive spirometry goal.

In some embodiments, the sensor is integrated as part of an air chamber within which the piston is displaced.

In some embodiments, the sensor is integrated as part of the piston.

In some embodiments, the sensor is remote from the processor, and the sensor operatively connects to the processor over one of a wired or wireless connection.

In some embodiments, the user interface, the sensor, the processor, and the display comprise a portable unit attachable to and detachable from an air chamber within which the piston is displaced.

In some embodiments, the incentive spirometry device further comprises a counter to count successful inspiring air events.

In some embodiments, the patient-specific incentive spirometry goal comprises a desired air volume of inspired air.

In some embodiments, the user interface comprises one or more switches enabling inputting of the desired volume of inspired air. In some embodiments, the one or more switches further enable bookmarking of at least one of an inspiring air event, a breathing event, and a patient state. In some embodiments, the incentive spirometry device further comprises a memory unit operatively connected to the processor, the memory unit storing the at least one of the inspiring air event, the breathing event, and the patient state. In some embodiments, the memory unit further stores at least one of minimum and maximum inspired/expired air volumes over the period of time or one or more subsets of the period of time.

Other features and aspects of the invention will become apparent from the following detailed description, taken in conjunction with the accompanying drawings, which illustrate, by way of example, the features in accordance with embodiments of the invention. The summary is not intended to limit the scope of the invention, which is defined solely by the claims attached hereto.

### BRIEF DESCRIPTION OF THE DRAWINGS

The present disclosure, in accordance with one or more various embodiments, is described in detail with reference to the following figures. The drawings are provided for purposes of illustration only, and merely depict typical or example embodiments. These drawings are provided to facilitate the reader's understanding of various embodiments and shall not be considered limiting of the breadth, scope, or applicability of the present disclosure. It should be noted that for clarity and ease of illustration these drawings are not necessarily made to scale.

FIG. 1 illustrates an example incentive spirometer.

FIG. 2A illustrates an example environment in which embodiments of the disclosure may be implemented.

FIG. 2B illustrates an example monitoring device according to embodiments of the disclosure.

FIG. 2C illustrates an example configuration of a monitoring device and incentive spirometer according to embodiments of the disclosure.

FIG. 2D illustrates an example configuration of a monitoring device and incentive spirometer according to embodiments of the disclosure.

FIG. 2E illustrates an example configuration of a monitoring device and incentive spirometer according to embodiments of the disclosure.

FIG. 3 is an operational flow diagram illustrating various operations that may be performed in accordance with embodiments of the disclosure.

FIGS. 4A and 4B illustrate example connections with supplementary computer devices in accordance with embodiments of the disclosure.

FIGS. 5A, 5B, and 5C depict another operational flow diagram illustrating various operations that may be performed in accordance with embodiments of the disclosure.

FIG. 6 is an example of a computing component that can be used in conjunction with various embodiments of the present disclosure.

The figures are not exhaustive and do not limit the present disclosure to the precise form disclosed.

### DETAILED DESCRIPTION

Embodiments of the present disclosure are directed to systems and methods for monitoring incentive spirometry through a device to facilitate self-administration and patient participation. In some embodiments, a device may be portable and, e.g., re-usable between multiple incentive spirometers. In some embodiments, such a device may be more permanently affixed to and/or integrated as part of an incentive spirometer. Incentive spirometry is designed to expand lung capacity by forcing a user to breathe sustained deep breaths. Use of an incentive spirometer is often prescribed by medical personnel following surgery to prevent post-operative complications, as well as for many respiratory diseases (e.g., pneumonia). As used herein, the term “incentive spirometry” refers to spirometry performed by an individual using an incentive spirometer.

The effectiveness of incentive spirometry for the prevention of postoperative pulmonary complications, or for hindering the progression of many respiratory diseases, is dependent on thorough provider instruction and sustained patient participation. Providers agree that best results are obtained when the device is used consistently. Insufficient self-administration can prevent the resolution of complications leading to prolonged hospital stays, high readmission rates (that are not reimbursed by insurance or hospital), and increased healthcare costs. Incentive spirometers with visual and auditory markers were introduced recently to encourage patient participation, but have had little effect. Furthermore, as hospital admissions increase, medical personnel have less time to spend with each patient to monitor regular incentive spirometer use. Indeed, the emergence of transmittable diseases, viruses, etc. via airborne transmission that can severely affect breathing make incentive spirometry more crucial than ever. Accordingly, there is a need in the art for a device capable of tracking patient incentive spirometer use and providing feedback that encourages further use. The systems and methods disclosed herein provide a way to meet this need by efficiently tracking user compliance, and by notifying medical personnel of that achievement.

Embodiments of the technology discussed herein provide a mechanism for efficiently tracking user compliance, and notifying medical personnel of that compliance. User compliance may be monitored by a monitoring device that tracks the inspiratory/expiratory metrics by a user, processes that information by comparing it to a desired air volume inputted by medical personnel, and displays a visual indication of results of the comparison. Such information may also be logged or stored for later use, e.g., analysis. The term “medical personnel” as used herein includes nurses, doctors, physician assistants, researchers, or other persons monitoring incentive spirometry.

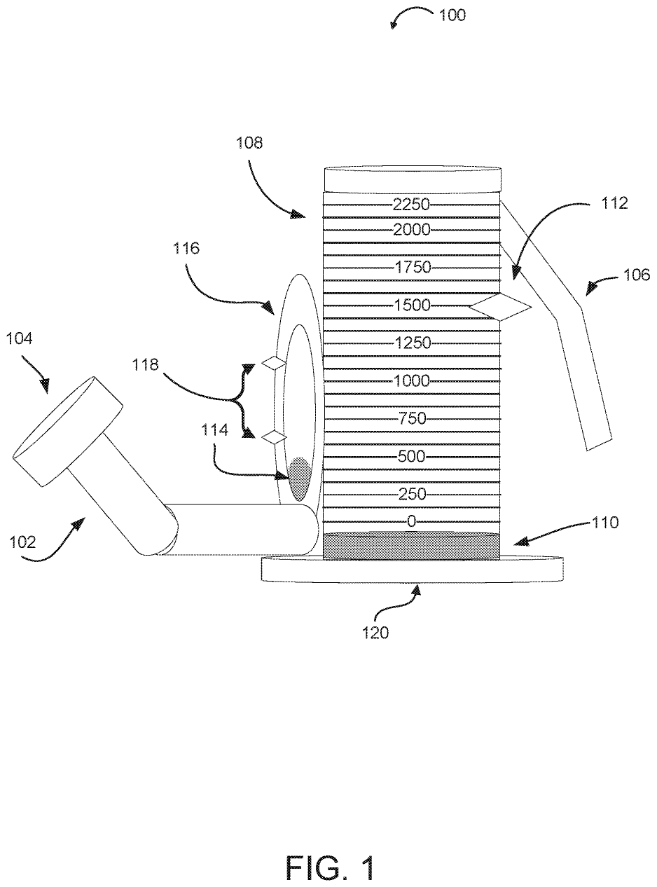

### FIG. 1

FIG. 1 depicts an example incentive spirometer 100 within the prior art. The technology disclosed herein enables greater functionality for medical professionals and users. As illustrated, incentive spirometer 100 may include, for example, an inhalation tube 102 with a mouthpiece 104, a handle 106, a base 120, an air chamber 108, a piston 110 within the air chamber 108, an adjustable marker 112, an indicator 114 inside a separate chamber 116 to indicate whether the user is inhaling too rapidly, and the base 120.

When a user inspires through the inhalation tube 102 (via the mouthpiece 104 in the illustrated example), the piston 110 within the air chamber 108 rises, indicating a volume of air the user inspired. During an exhale breathing event, when a user expires through the inhalation tube 102, the piston 110 rises, indicating a volume of air expired into the air chamber 108. The volume of air inspired (or volume of air expired), as used herein, is the total volume of air inhaled into (or exhaled from) the lungs during a single breath. The adjustable marker 112 may be positioned to indicate, for example, the volume of air the user should reach/attain through inspiration (or expiration) when using the device. The adjustable marker 112 may also indicate, for example, the maximum volume of air achieved by the user through inspiration (or expiration). The separate chamber 116 located next to the air chamber 108 identifies to the user whether he or she is inhaling (or exhaling) too quickly by moving the indicator 114 housed within the separate chamber 116 upwards or downwards. Markers 118 located on the outside of the separate chamber 116 provide guidance to the user, so that the he or she may inspire (or exhale) at a constant or otherwise desired rate.

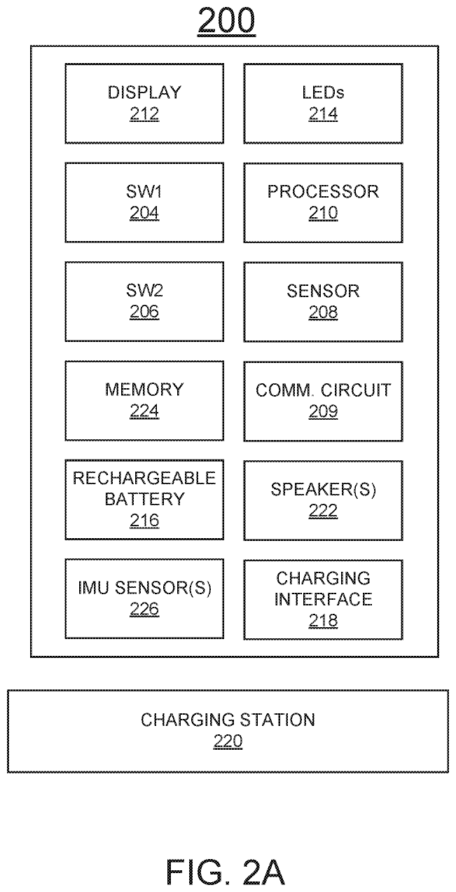

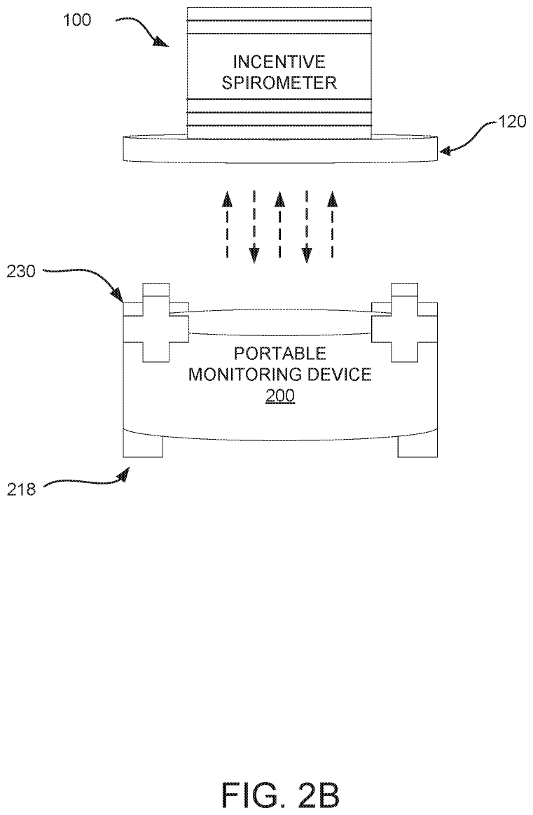

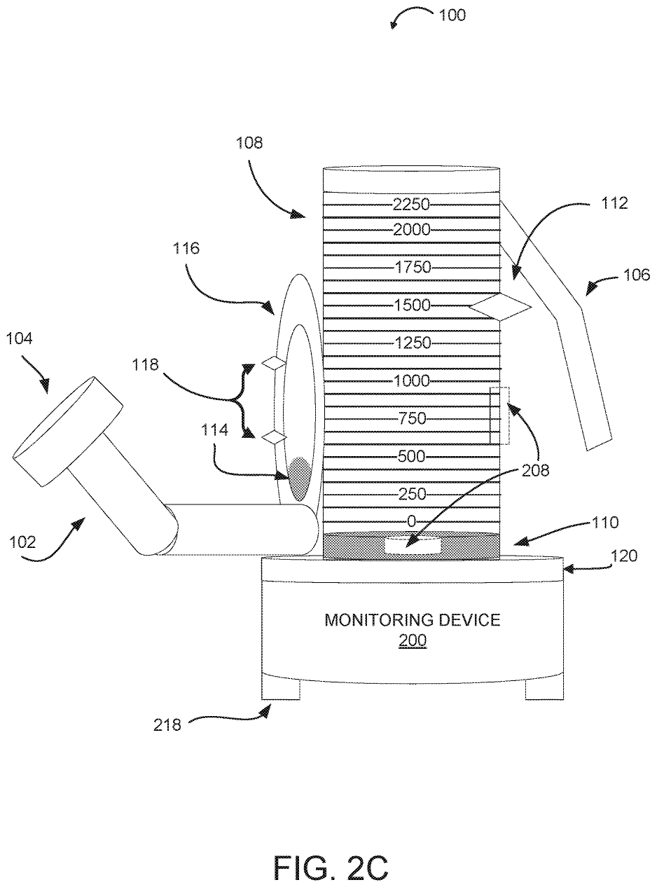

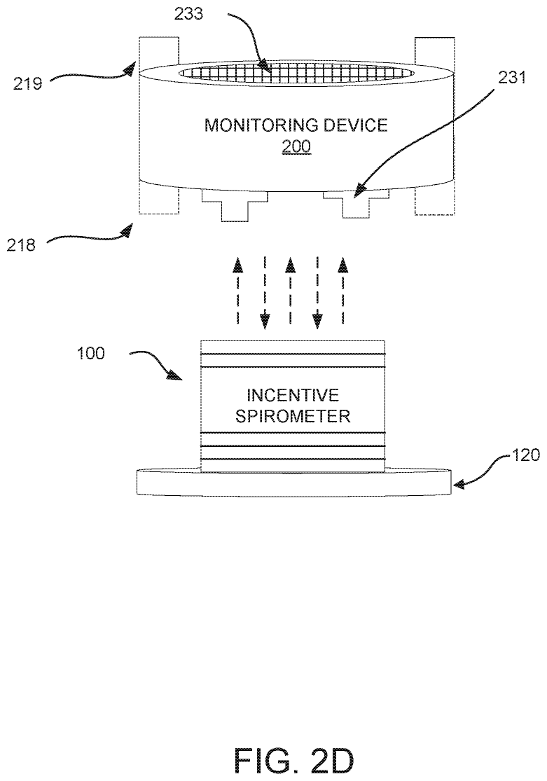

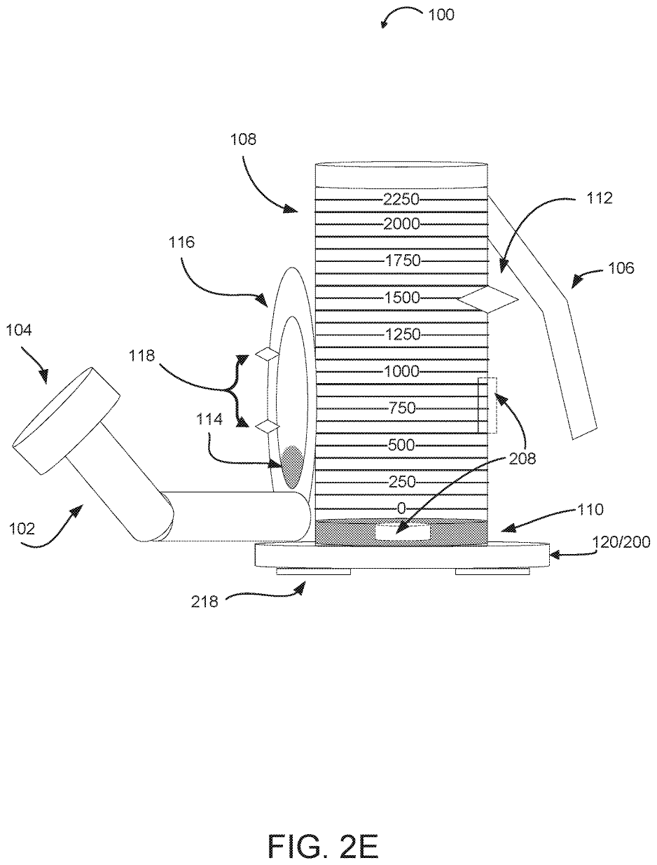

### FIGS. 2A-2E

FIG. 2A is a block diagram illustrating an example monitoring device 200 in accordance with the technology disclosed herein. The monitoring device 200 enables medical professionals to ensure patient compliance using the incentive spirometer 100 at the device itself, eliminating the need for the communication of raw data to a networked system for determination. As illustrated, the monitoring device 200 in various embodiments may include a first switch 204, a second switch 206, a sensor 208, a processor 210, an display 212, a plurality of LEDs 214, a rechargeable battery 216, charging interface 218, speaker(s) 222, inertial measurement unit (IMU) sensors 226, and a charging station 220 to which the monitoring device 200 may be connected.

The first switch 204 and second switch 206 may be operatively connected to a processor 210. The first switch 204 and second switch 206 may comprise buttons, toggles, or any other components capable of receiving input (e.g., tactile input) from a user. In various embodiments, the first switch 204 and second switch 206 may be used by medical personnel to input, for example, a desired air volume based on the user. As used herein, the term “desired air volume” (or predicted goal, or goal) means a standardized air volume based on the individual user's sex, weight, height, age, and/or other parameters specific to that individual user (e.g., past surgeries, smoking history, workout history, diabetes, history of cancer, other illnesses, etc.). In various embodiments, the desired air volume may be an air volume between the range of about 250 ml to about 3000 mL. The desired air volume could be represented as a range of volumes (for example, between about 450 ml and 475 mL) or discrete volume with some tolerance for variation in achieving the desire air volume (for example, about 550 mL).

In various embodiments, medical personnel can use the first switch 204 to increment the desired air volume, and the second switch 206 allows a medical personnel to decrement the desired air volume. In embodiments, the first switch 204 and second switch 206 may increment or decrement the desired air volume by a range between about 1 mL to about 1000 mL, or from about 200 ml to about 2000 mL. In various embodiments, the desired air volume may be incremented or decremented by a range between about 250 ml to about 500 mL.

The amount of change caused by incrementing or decrementing may vary between embodiments, depending on the level of precision required. For example, the desired air volume may be incremented by 50 mL at a time in some embodiments, or 1 ml at a time in other embodiments. A person of ordinary skill in the art would know how to vary the amount of increase or decrease based on the needs of a particular implementation, and the examples provided should not be interpreted as limiting the subject matter of this disclosure to any particular amount of variation in the increment or decrement.

The first switch 204 and the second switch 206 may be pressed at the same time in some embodiments to reset the desired air volume. Pressing the first switch 204 and the second switch 206 simultaneously in such situations begins a command function to reset the desired air volume. Pressing the first switch 204 and the second switch 206 simultaneously sends a signal to the processor 210, which is operatively connected to the first switch 204 and second switch 206, to initiate a command whereby the processor 210 sends a signal to the LEDs 214, which are operatively connected to the processor 210. Once the command is received by the LEDs 214, the LEDs 214 will begin blinking and the desired air volume may then be inputted. If the LEDs 214 do not blink, the first switch 204 and second switch 206 may be simultaneously pressed again to resend the command. In embodiments, once the desired air volume is set, the first switch 204 and the second switch 206 may then be pressed and held simultaneously to set the new desired air volume.

In embodiments, the first switch 204 and second switch 206 may be pressed simultaneously to reset the elapsed time. The reset of elapsed time in this manner may occur simultaneously with resetting the desired air volume as discussed above, while other embodiments may have the first time the switches are pressed at the same time reset the desired air volume and a second time the switches are pressed at the same time resets the elapsed time. In other embodiments, a time reset switch (not pictured) may be included to reset the elapsed time.

The switches 204, 206 may be configured to perform a variety of functions of the monitoring device 200, some of which are discussed above. In various embodiments, the switches 204, 206 may be configured, individually or in combination, to perform one or more of the following functions: increment/decrement desired air volume; set the minimum desired air volume; set one or more thresholds; control the display 212; initiate a transfer of data from the monitoring device 200 to a remote location; among others. In various embodiments, additional switches may also be included in addition to the first switch 204 and second switch 206.

In various embodiments, the actions of switches 204, 206 may vary depending on the function to be performed. For example, in some embodiments one or more switches may be configured to increment a value when pressed, while holding the same switch may activate the configuration of thresholds. Non-limiting examples of the types of actions which the switches 204, 206 may perform include pushing, holding, toggling, twisting, among other depending on the type of switch implemented. In some embodiments, the switches 204, 206 may be different types of switches. For example, in some embodiments first switch 204 may be a toggle, capable of being flipped into one of two positions, while the second switch 206 is a rocker switch capable of being depressed in one or two directions continuously, returning to a neutral position after ever action. As another example, the first switch 204 may be a pushbutton, the second switch 206 a toggle, and a third switch (not pictured) is a rotary or dial. The type of switch may indicate the type of function a switch is capable of performing. The duration of switch actuation can also be used to toggle between operational modes or states of use, or to perform different functions. A person of ordinary skill in the art would understand the capabilities of different switches and would know what type of switch to implement to perform the various functions of the monitoring device 200 discussed above. It should be noted that the use of alternatives to physical switches are contemplated. That is, the functionality one or more of the switches described herein, e.g., switches 204, 206, can be embodied using other mechanisms, for example, such as voice-activated switches/mechanisms that can perform the aforementioned incrementing, decrementing, etc. vis-à-vis audio input.

Although examples of the functions of the first switch 204 and second switch 206 have been discussed with specific reference to each respective switch, a person of ordinary skill in the art would understand that the modifiers “first” and “second” do not connote any priority in positioning on the monitoring device 200, or in position relative to each other.

Referring still to FIG. 2A, the monitoring device 200 includes one or more sensors 208. The sensors 208 are operatively connected to the processor 210. In various embodiments, the sensors 208 may measure the inspired (or expired) air volume in the incentive spirometer 100 when the monitoring device 200 is connected to the incentive spirometer 100. In some embodiments, monitoring device 200 may be a portable device, where monitoring device 200 can be used by attaching to an existing incentive spirometer, e.g., incentive spirometer 100. For example, monitoring device 200 can be attached to a base 120 (as described below and with reference to FIG. 2B). After use, incentive spirometer 100 and monitoring device 200 may be separated, and monitoring device 200 may be attached to another incentive spirometer for use, and so on. In other embodiments monitoring device 200 may be permanently attached/affixed to an existing prior art incentive spirometer. In still other embodiments, the functionality of monitor device 200 (described herein) may be implemented/integrated into a novel incentive spirometry device.

In various embodiments, the sensors 208 measure inspired/expired air volume by tracking the time of flight of the piston 110 within the air chamber 108 of the incentive spirometer 100, when the monitoring device 200 is connected to the incentive spirometer 100. The sensors 208 may track the time of flight through, for example, a light signal, including light signals transmitted from ambient, infrared, laser, or other light emitting sources. The sensors 208 may also track time of flight through, for example, a sound signal. Once the inspired/expired air volume has been received by the sensors 208, the volumetric measurement of air may then be relayed to the processor 210. It is to be appreciated that the sensors 208 may track a plurality of time of flight measurements.

The sensors 208 may further include sensors for detecting a velocity at which air is inputted (i.e. through inhalation/inspiration) into the incentive spirometer 100. In various embodiments, the sensors 208 may include sensors for detecting an acceleration of the piston within the incentive spirometer 100.

Monitoring device 200 may include IMU sensor(s) 226 for detecting motion of monitoring device 200 by measuring a specific force, angular rate, and orientation of the monitoring device 200. The IMU sensors 226 may be implemented as accelerometers for measuring linear acceleration of monitoring device 200, one or more gyroscopes for measuring angular velocity, one or more magnetometers, or the like or combinations thereof. IMU sensors 226 may provide time-series data of movements, which processor 210 can use to detect movement and orientation of monitoring device 200.

In some cases, data obtained from IMU sensor(s) 226 can be used to detect compliance with using monitoring device 200. For example, processor 210 may use data from IMU sensor(s) 226 to detect that the monitoring device 200 is being moved and/or orientated so as to mimic a successful attempt. As an illustrative example, a patient may attempt to flip incentive spirometer 100 connected to monitoring device 200 upside down so as to cause piston 110 to fall below the desired air volume, thereby mimicking an attempt. In this case, IMU sensor(s) 226 can be used to measure the motion, which processor 210 can utilize to detect that monitoring device 200 was been used improperly. As a result, processor 210 can register an unsuccessful attempt and that the use was not in compliance.

Data obtained from IMU sensor(s) 226 can also be used to detect usage of monitoring device 200, which can be used to trigger an auto on/off functionality of monitoring device 200. For example, IMU sensor(s) 226 can detect movement exerted on the monitoring device 200, which processor 210 may utilize to recognize as a patient or other user picking up the device. Responsive to recognizing the usage, processor 210 can trigger rechargeable battery 216 to supply power to the monitoring device 200 and automatically turn on the other components or elements of monitoring device 200. Conversely, data obtained by IMU sensor(s) 226 can be used to detect that monitoring device 200 has been placed down and is no longer in use, for example, by detecting that monitoring device 200 has been placed on a flat surface or connected to charging station 220. Responsive to this detection, processor 210 can cause rechargeable battery 216 to stop supplying power to certain components or elements of monitoring device 200 so as to automatically power down monitoring device 200. Powering down of monitoring device 200 may include a system wide shut off or a sleep mode in which standby power is supplied to certain components or elements (e.g., processor 210, IMU sensor(s) 226, and/or memory 224). Thus, IMU sensor(s) 226 can be utilized to conserver power consumption through triggering of auto on/off functionality.

The monitoring device 200 may also include a processor 210. The processor 210 may include circuits, such as logic or other circuits for one or more of receiving, processing, and/or storing content, data, or other information. The circuits may facilitate the receipt (e.g., as data input) of such content, data, or other information, as well as the generation of such content, data, or other information by the monitoring device 200. The circuits may further facilitate the transmission or delivery of such content, data or other information by the monitoring device 200. In some embodiments, as will be described below, e.g., when sensors 208 are located remotely from monitoring device 200, sensors 208 may communicate sensed information/data, e.g., measured inspired/expired air volume (whether measured/sensed directly or corresponding information/data that processor 210 may convert or translate into inspired/expired air volume), inspired air velocity, etc., to monitoring device 200. Accordingly, monitoring device 200 may include communications circuitry 209, such as a transmitter/receiver/antenna for communicating with such sensors 208. In embodiments, the processor 210 may receive the desired air volume from the first switch 204 and second switch 206, and may also receive detected data form the sensors 208. In various embodiments, the processor 210 receives a plurality of time of flight measurements from the sensors 208. In embodiments, the processor 210 converts the time of flight measurement into an inspired/expired air volume. In embodiments, the processor 210 converts a plurality of time of flight measurements into inspired/expired air volumes. The processor 210 may also receive other types of sensed data, such as the velocity of air input into the incentive spirometer and the period in which air was inputted into the incentive spirometer. The processor 210 can include logic to analyze the data received from the first switch 204, the second switch 206, the sensors 208, and other components of the monitoring device 200 to calculate various metrics for inspiratory and expiratory breathing events, referred to herein as inspiratory/expiratory metrics. Example inspiratory/expiratory metrics include, but are not limited to: number of attempts; whether a patient succeeded per the parameters; compliance; maximum inspired air volume (i.e., max volume); minimum inspired air volume (i.e., min volume); acceleration of the piston; total time air input was within recommended levels; percentage of time spent within the acceptable ranges for a success; inhalation rate (e.g., volume of inspired air over a defined timeframe); force expiratory volume (FEV) (i.e., maximum volume of air exhaled during a force breath over a period of time); forced vital capacity (FVC) (i.e., total amount of air exhaled over a period of time); peak expiratory flow rate (PEFR) (i.e., maximum volume of air forcefully expelled from the lungs in one quick exhalation, and can be an indicator of ventilation adequacy as well as airflow obstruction); among others. As described herein, an “attempt” is registered when the piston 110 reaches a certain threshold in the air chamber 108. For example, the threshold may be entered as 250 ml; if the piston 110 reaches or exceeds 250 ml in the air chamber 108, an attempt is registered. In some implementations, the threshold may be set to count each breath event as an attempt. In an example, a threshold may be set as a prescribed (e.g., target) tidal volume. As described herein, “success” is registered when piston 110 in the air chamber 108 reaches (i.e., equals) or exceeds the threshold and desired air volume input by medical personnel. For example, the desired air volume may be indicated by adjustable marker 112. As used herein, “compliance” is the summation of successes over a period of time (t). By registering a “success” as reaching or exceeding the threshold, double inhalations can be ignored, while monitoring for successful single inhalation.

As described herein, the terms “maximum inspired air volume,” “maximum expired air volume,” or “max volume,” refer to a volume of air associated with the highest point that piston 108 reaches. In various embodiments, max volume may be tracked over a period of time. For example, the highest point that piston 108 reaches over a period of, for example, 5 minutes, is the max volume. As described herein, the terms “minimum inspired air volume,” or “min volume,” refer to the volume of air associated with the lowest point that piston 108 reaches. In various embodiments, min volume may be tracked over a period of time. In various embodiments, the processor 210 may present a visual output of the processed inspired/expired air volume(s), as well as other processed information (e.g., the desired air volume) through the LEDs 214 and/or display 212.

Communications circuitry 209 may also provide for transfer/receipt of data between monitoring device 200 and external devices. Examples of communications circuitry 209 might include a modem or softmodem, a network interface (such as an Ethernet, network interface card, WiMedia, IEEE 802.XX or other interface), a communications port (such as for example, a USB port, IR port, RS232 port Bluetooth® interface, or other port), or other communications interface circuitry. Data exchanges via communications circuitry 209 might typically be carried on signals, which can be electronic, electromagnetic (which includes optical) or other signals capable of being exchanged by a given communications circuitry 209. These signals might be provided to communications circuitry 209 via a channel. This channel might carry signals and might be implemented using a wired or wireless communication medium. Some examples of a channel might include a phone line, a cellular link, an RF link, an optical link, a network interface, a local or wide area network, and other wired or wireless communications channels. Further, the channel through which data is exchanged by communications circuitry 209 may be implemented using various wireless standards, such as, but not limited to, Bluetooth, Wi-Fi, 3GPP standards (e.g., 2G GSM/GPRS/EDGE, 3G UMTS/CDMA2000, 4G LTE/LTE-U/LTE-A, 5G), and the like.

Monitoring device 200 may further include a memory 224 for storing thresholds, desired air volumes, inspired/expired air volumes, and any other various measurements sensed, monitored, or otherwise obtained through use of monitoring device 200. In various embodiments, the memory 224 may be a fixed or removable storage medium, such as, for example, a hard disk drive, a solid state drive, a magnetic tape drive, an optical disk drive, a compact disc (CD) or digital video disc (DVD) drive, flash memory, USB memory, or other form of fixed or removable storage medium. The type of storage medium may be dictated on the particular implementation, based on performance and/or form factor requirements that a person of ordinary skill in the art would understand and know how to select the storage medium that is appropriate. In various embodiments, the memory 224 may be computer-readable medium having stored therein computer software or data for performing the various functions of the monitoring device 200.

It should be noted that the collection or logging or calculation of data can be effectuated through storage of monitored signals from sensors 208, calculated information via processor 210, and so on. The data can be registered to a respiratory database and associated with a particular patient within the respiratory database corresponding to the data, as will be described below in greater detail. In embodiments, the collection or calculation of data can be performed by components monitoring device 200, e.g., sensors 208 directly. That is, sensors 208 may have their own memory or data caches/buffers in which such data may be stored, and later retrieved. In embodiments, and in addition to the aforementioned usage of first and second switches 204 and 206, one or more of first and second switches 204 and 206 can be used to retrieve collected or calculated data stored in memory 224. In some embodiments sensors 208 may have their own respective interfaces with which a user or connected processor, e.g., laptop computer, smart phone, dedicated medical device, can retrieve stored or logged data.

In embodiments, the monitoring device 200 such as the one depicted in FIG. 2A may include a display 212. Non-limiting examples of display 212 include: a liquid-crystal display (LCD); an organic LCD (OLCD); a light emitting diode display (LED); an organic light emitting diode display (OLED); digital light processing display (DLP); among others. The display 212 is operatively connected to the processor 210, and receives an information from the processor 210 that may be displayed to the user. The information may be displayed to the user in real-time with measuring inspiratory/expiratory metrics. Providing real-time display of such information may incentivize a patient to use monitoring device 200 and improve compliance. Such information may include one or more of the following: desired air volume(s), inspired/expired air volume(s) (e.g., attempts, successes, compliance, maximum inspired air volume, minimum inspired air volume, FEV, FCV, PEFR, etc.); time elapsed (in units of hours, minutes, seconds, etc.); or other information that may be useful to the user or medical personnel (e.g., flow rate).

As noted above, information can be logged or stored in memory 224, sensors 208 themselves, etc. The logged or stored information can be made accessible via a user interface. In some embodiments, a user may specify parameters according to which such information is logged/stored. For example, information displayed to a user can include maximum/minimum inspired/expired air volumes. A user may specify that such maximum/minimum inspired/expired air volumes are to be recorded or logged in accordance with specified time parameters, e.g., the maximum/minimum inspired/expired air volumes every 10 minutes or only if such information exceeds or falls below a given threshold, etc. It should be understood that data/information collection in accordance with embodiments can be customized in accordance with desired monitoring needs/desires of medical personnel. In some embodiments, medical personnel use switches 204 or 206 (or another interface(s)) to specify that only minimum inspired/expired air volume is to be recorded. It should be understood that first and second switches 204 or 206 may transition from a programming/configuration mode (where a user can use such switches to specify thresholds) to a data retrieval mode (where a user can use first or second switches 204/206 to pull up logged information for perusal/review via display 212).

In still other embodiments, a patient may use switches 204 or 206 to “bookmark” certain events or states to be recorded with the logged information. For example, during use, first or second switches 204 or 206 may be actuated by a patient to indicate some sort of breathing event or patient state, e.g., during use a patient may experience difficulty breathing, in which case, the patient may toggle or depress one of first or second switches 204 or 206, the actuation of which is recorded. During information retrieval, that bookmark can be presented along with the other recorded information (rate of airflow, maximum/minimum inspired air volume, FEV, FCV, PEFR, etc.) so that medical personnel may be made aware that the patient experienced some event/was in some state of respiratory distress/discomfort at a particular time of usage of incentive spirometer 100.

As noted above, information can be displayed in real-time on display 212. In some embodiments, a user may specify parameters according to which such information is displayed in real-time. For example, information displayed to a user can include maximum/minimum inspired/expired air volumes. Maximum/minimum inspired/expired air volumes can be displayed via display 212 in real-time during use of an incentive spirometer 100 that is connected to monitoring device 200, e.g., the maximum/minimum inspired/expired air volumes for each attempt or over a set period of time (e.g., 10 mins), average inspired/expired air volumes, indication that such information exceeds or falls below a given threshold, etc. It should be understood that data/information collection in accordance with embodiments can be customized in accordance with desired monitoring needs/desires of medical personnel. The real-time display of this information may incentivize a patient to continue to use the incentive spirometer 100 through real-time monitoring of compliance. In some embodiments, medical personnel using switches 204 or 206 (or another interface(s)) to specify that only minimum inspired/expired air volume is to be recorded.

In various embodiments, a user or medical personnel may interact with the display 212 screen. By way of example, such an display 212 may be a touchscreen that accepts various hand gestures as inputs through a graphical user interface.

As further depicted in FIG. 2A, embodiments of the monitoring device 200 may include a plurality of LEDs 214 (i.e., light emitting diode). The LEDs 214 are operatively connected to the processor 210, and receive information from the processor 210 that may be displayed to the user and medical personnel. The LEDs 214 may be any one or more of the following colors: red, green, blue, yellow, purple, white, black, and brown, or any combination thereof of RGB. Let it be appreciated that the list is not meant to be exhaustive, and more colors than the ones mentioned may be used. In various embodiments, the LEDs 214 are colored differently, where a color signifies certain information to the user and medical personnel. For example, in embodiments, the monitoring device may include two differently colored LEDs 214. One of the LEDs 214 may be, for example, a red LED and may signify to the user and medical personnel that the desired air volume input has not been achieved, while the other LED may be, for example, a green LED and may signify to the user and medical personnel that the desired air volume has been met. In embodiments, different combinations of colors may be used to signify different achievements.

In various embodiments, the LEDs 214 may be configured to indicate whether a patient has continued to comply with a recommended use over time. For example, an LED 214 may remain green during the period where the patient has been using the incentive spirometer 100 as required, but turn another color if the patient fails to use the incentive spirometer as many times as necessary. In this way, medical personnel can see whether a patient is using the incentive spirometer as scheduled, regardless of whether the patient has achieved the desired levels of performance.

The information represented by LEDs 214 discussed above could also be displayed on the display 212 in various embodiments.

Embodiments of the monitoring device may also include a rechargeable battery 216. The rechargeable battery 216 is operatively connected by circuitry to one or more components of the monitoring device 200 for supplying electric power. In embodiments, the rechargeable battery 216 is recharged when operatively connected to the charging station 220. Charging interface 218 located on one face of the monitoring device 200 interact with the charging station 220 to receive the electrical current. In some examples, charging interface 218 may be implemented as charging pins, one or more charging ports (e.g., Universal Serial Bus (USB) type ports), wireless charging coil, and the like. The rechargeable battery 216 may be consisted of any one of the following types: nickel cadmium, nickel-metal hybrid, lead acid, lithium ion, or lithium polymer. The rechargeable battery 216 may be configured to hold a charge capable of providing continuous operation of the monitoring device 200 (e.g., 24 hours of operation or more). According to various examples, the monitoring device is designed and manufactured according to standards set forth by the U.S. Food and Drug Administration and the Federal Communication Commission, as well as complying with IEC 60601 standard for electrical safety and electromagnetic compatibility.

The monitoring device 200 may also include speaker(s) 222 for audio outputs. In some implementations, the information represented by LEDs 214 discussed above could also be output as audio notifications through speaker(s) 222. For example, unique tones or sounds may be assigned to meeting certain thresholds. As an illustrative example, a first sound may be output by speaker(s) 222 during a period where the patient has been using incentive spirometer 100 as required, but a second sound if the patient fails to use incentive spirometer 100 as required. A different sound may signify to the user and medical personnel that the desired air volume input has not been achieved, while yet another sound may signify to the user and medical personnel that the desired air volume has been met. In embodiments, different sounds may be used to signify different achievements. In some cases, instructions may be provided as positive reinforcement triggered when different achievements are met by a patient, such as when a desired air volume input is reached and a congratulatory instructions can be communicated to the patient. Different positive reinforcement instructions may be output based on different achievements.

In another example, speaker(s) 222 may be implemented to communicate patient coaching and therapy to the patient based on different achievements. The speaker(s) 222 may output spoken words and sentences to the patient that instruct the patient on certain usage to incentive different achievements. For example, instructions to use the incentive spirometer 100 as required can be output by speaker(s) 222 or to use incentive spirometer 100 more frequently. As another example, instructions may be output that explain steps to achieve a desired air volume input. The instructions may be prerecorded by a health care provider and stored in memory 224. Recorded instructions could then be retrieved from memory 224 and output from speaker(s) 222 according to measured metrics. In another example, an artificial intelligence and machine learning algorithm may be implemented to generate context specific instructions according to measured metrics, which can be converted from text to speech algorithm and output from speaker(s) 222. In some implementations, monitoring device 200 may be communicatively coupled to a virtual assistant technology (e.g., Amazon Alexa, Apple Inc.'s Siri, Google Assistant or the like) via communications circuitry 209, which may be utilized to output instructions to a patient through an external device. Additionally, virtual assistant technology can be utilized to communicate notifications and reminders to a patient.

In yet another implementation, instructions may be supplied by a health care provider in real-time based on a real-time usage of incentive spirometer 100. For example, monitoring device 200 may measure inspire/expire metrics as described above, which can be communicated to an external device at a geographically separate location from monitoring device 200, such as a nursing station or other health care provider. The health care provider at the separate location may then speak instructions that are transmitted to monitoring device 200 and output from speaker(s) 222.

The monitoring device 200 may implement gamification techniques to motivate, incentivize, and engage patients to use incentive spirometer 100 to obtain different achievements. For example, processor 210 may implement gamification techniques stored in memory 224 to incentivize a patient's usage compliance with incentive spirometer 100 according to different achievements. Memory 224 may store rewards for patients with hierarchical tiered achievements measured inspired/expired metrics. Rewards may be provided as points, achievement badges, progress bars, virtual currency or the like. Current rewards achieved by a user may be displayed on display 212, as well as a next reward and criteria for achieving the next reward. As an example, certain rewards may be attributed to increments of attempts made over a period of time, for example, a first reward for once a day, a second reward for 10 times a day, a third reward for a n-number of consecutive days of use. In another example, rewards may be based on achieving a desired air volume input or other inspire/expire metric.

As another example, gamification can be implemented by monitoring device 200 by making breathing events feel like games by adding a narrative or progress bar to each breathing event. For example, a progress bar may be progressively filled in according to a level of volume of air the inspired. That is, as the piston 110 increase in accordance with the volume of air inspired, a progress bar may be filled in to represent how close to the desired air volume input level the piston 110 is. The progress bar may be displayed on display 212 or an external device for the patient to use to incentivize the breathing event. The progress bar may be a simple bar that is filled in or other graphical implementations, such as a container of liquid that is filled to a limit and overflows when the limit is reached, a graphically rendered horse (or other animal) that is displayed as running at a speed corresponding the volume of air inspired, or any graphical representation thereof.

The monitoring device 200 or components/features thereof may be implemented in combination with, or as an alternative to, other devices/features/components described herein, such as those described with reference to other embodiments and figures. The monitoring device 200 may additionally be utilized in any of the for methods making and/or using such devices/components/features described herein. The monitoring device 200 may also be used in various applications and/or permutations, which may or may not be noted in the illustrative embodiments described herein. For example, one or more components or elements of monitoring device 200 may be implemented remotely or separately from each other. One or more sensors 208 may be located remotely from processor 210 in some embodiments. Display 212, LEDs 214, switches 204/206, etc. may be remotely located from each other or other components or elements. For example, rather than positioning switches at a bottom surface or base of monitoring device 200, switches 204/206 may be implemented on a base 120 of incentive spirometer 100. Again, such embodiments are merely examples, and not meant to be limiting. One of ordinary skill in the art would understand how to implement the functionality of each of the components or elements of monitoring device 200 in various ways.

Furthermore, one or more components or elements of monitoring device 200 may be implemented in charging station 220. For example, display 212, LEDs 214, switches 204/206, speaker(s) 222, etc. may be part of charging station 220. In some embodiments, one or more components or elements of monitoring device 200 maybe be implemented in both charging station 220 and monitoring device 200. The components or elements included in the charging station 220 may execute similar functionality to corresponding components or elements described in above. Furthermore, charging station 220 may comprise a memory (e.g., similar to memory 224) for collecting and logging information stored on monitoring device 200 when the monitoring device 200 is connected to charging station 220, whether wireless or through a physical connection. Thus, in some embodiments, memory 224 may provide for temporary storage of information, which can be supplied to a memory of charging station 220 for permanent storage and/or transferring to an external device through a communication circuit.

Charging station 220 may include a communication circuit similar to communications circuitry 209, as well as a charging interface adapted to supply power to charging interface 218. Charging interface of charging station 220 may be adapted to electrically connect to charging interface 218 via a wireless or physical connection. Through communication circuit, two-way communication and exchange of information can be established between charging station 220 and monitoring device 200, as well as external devices. Information from monitoring device 200 can be stored and displayed on charging station 220 via a display, LED, etc.

As illustrated in FIG. 2B, the monitoring device 200 may, in some embodiments, be operationally coupled to the base 120 of an incentive spirometer 100 through a wireless connection or physical attachment. The monitoring device 200 may have a top end, and a bottom end, and has a face capable a displaying through a visual indication, a plurality of user information. In embodiments, one face of the monitoring device 200 has charging interface 218 for charging the device via charging station 220. In some embodiments, charging interface 218 implemented as a wireless charging coil may be adapted to receive charge wireless from any wireless charging pad, such as charging station 220 implemented with a wireless charging coil or any other wireless charging pads. In other embodiments, alone or in combination, one or more charging ports (not pictured) may be included to charge the monitoring device 200 that can be electrically connected to an electrical source, such as a socket, via a wired connection (e.g., a charging cable).

As illustrated, monitoring device 200 may include connectors 230 that are configured to mate with the base 120 of the incentive spirometer 100. The top of the monitoring device 200 may be cylindrical in shape, or may be round, square, rectangular, or a combination thereof. The connectors 230 may be mechanical and may include, for example, screws, latches, Velcro, locks, snaps, buttons, magnets, or some combination thereof. In various embodiments, the connectors 230 may be adjustable components, allowing the monitoring device 200 to mate with various different incentive spirometers that may have different shaped bases. The connectors 230 may comprise any suitable component for connecting to a generic incentive spirometer. For example, in some embodiments the connectors 230 may be elbow-like latches comprising an arm configured to clamp onto the base 120 and apply pressure in a downward direction to secure the base 120 to the top of the monitoring device 200. As another example, the connectors 230 may comprise component pairs configured to secure the base 120 of the incentive spirometer 100 to the top of the monitoring device 200, such as a strap and locking mechanism. A person of ordinary skill in the art would understand that a variety of different mechanisms may be used as connectors 230 and that the examples above are not meant to be limiting.

While FIG. 2B illustrates incentive spirometer 100 operationally coupled to monitoring device 200, any incentive spirometer may be adapted to operatively couple to monitoring device 200 by adapting a base of an incentive spirometer to attach to the monitoring device 200.

By utilizing a monitoring device 200 in accordance with the technology disclosed herein, medical personnel are capable of obtaining results indicative of a patient's progress at the incentive spirometer, without the need to transfer the data to another location. Moreover, medical personnel have greater flexibility in administering therapy through greater control over setting parameters and ensuring patient participation. The ability of connectors 230 to mate with a variety of different bases provides medical personnel with a single tool that can be operatively couple to augment any type of incentive spirometer on hand. This lowers cost by facilitating the collection of relevant data for medical personnel regardless of the incentive spirometer used by the patient.

As alluded to above, it should be understood that sensors 208 may be positioned within or about one or more areas of incentive spirometer 100. That is, rather than incorporating sensors 208 as part of monitoring device 200, sensors 208 may be remotely implemented. For example, sensors 208 may comprise their own communication circuits, mechanisms, and the like so that sensors 208 may communicate, e.g., wirelessly or via physical wired connection with a processor of monitoring device 200, e.g., processor 210 (described in greater detail below). Accordingly, a sensor(s) 208 may be disposed on an outer or inner surface of air chamber 108. Alternatively, or additionally, a sensor(s) 208 may be disposed on or within/as part of piston 110 to track time of flight, inspired air velocity, etc. Information or data sensed by sensors 208 may then be relayed to monitoring device 200 for use or processing as described herein. It should also be understood that the aforementioned embodiments are not meant to be limiting in any way, and sensors 208 can be incorporated in a variety of ways to gauge a patient's use of incentive spirometer 100.

For example, and as illustrated in FIG. 2C, monitoring device 200 (which may be a portable monitoring device or fixed monitoring device) may be operatively connected to or integrated as part of incentive spirometer 100. As further illustrated, one or more sensors 208 may be positioned on/within/about one or more areas of incentive spirometer 100, e.g., on an outer/inner (or both) surface(s) of air chamber 108, in or as part of piston 110, etc. Sensing functionality may be implemented as part of air chamber 108, where some or all of an inner/outer surface(s) of air chamber 108 may act as a sensor capable of sensing movement of piston 110 therein. For example, air chamber 108 may comprise a cylinder position sensor that provides position information regarding the movement of the piston 110 therein. In still other embodiments, piston 110 may be a sensor in and of itself, rather than incorporating a distinct sensing device thereon or therein. For example, piston 110 can be embodied as a linear position sensor, linear displacement transducer, and the like.

Sensors 208 may relay sensed information, e.g., motion of piston 110, air velocity through air chamber 108, etc. through resident communication circuitry to monitoring device 200, e.g., a processor 210 of monitoring device 200 via a communications circuitry 209, which may comprise a transceiver, one or more antennas, one or more wired connections/ports, and so on. In this way, the functionality of monitoring device 200 need not necessarily be contained as or in a single unit/device.

In some embodiments, monitoring device 200 may be provided as a kit including one or more remote sensors that a user, e.g., patient, or health care provider may place (via magnet, temporary or permanent adhesive, velcro, etc.) in a convenient location on/in incentive spirometer 100. In this way, different types of incentive spirometers may be imbued with the monitoring functionality described herein.

Indeed, as illustrated in FIG. 2D, and as alluded to above, monitoring device 200 may be operatively couple to or used with incentive spirometer 100 in a variety of ways. In some embodiments, rather than attachment to a base 120 of incentive spirometer 100, monitoring device 200 may be operatively connected to a top (or other section(s)) of incentive spirometer 100 such as atop air chamber 108.

In such an embodiment, one or more connectors 231 may facilitate permanent or temporary operative attachment to incentive spirometer 100. For example, connectors 231 may comprise any one of or combination of the aforementioned connectors. For example, connectors 231 may comprise friction-fit tabs, protrusions, gripping elements, etc. that may retain monitoring device 200 atop air chamber 108.

To enable proper air flow through incentive spirometer 100, monitoring device, in some embodiments, may include one or more vents, ports, or other openings 233 allowing for the passage of air through incentive spirometer 100 during use. It should be noted that one or more components of monitoring device 200 may be remotely located. In some embodiments, the one or more components of monitoring device 200 may be positioned so as not to impact the operation of incentive spirometer 100/monitoring device 200. For example, components of monitoring device 200 may be positioned along one or more edges of monitoring device 200 containing, e.g., a printed circuit board that can be flexible or shaped to accommodate such placement of monitoring device 200's components, or otherwise spaced in a manner to allow openings 233 to be implemented.

Charging interface 219 may be the same/similar to charging interface 218 described above, except charging interface 219 may be implemented on an opposite side or surface of monitoring device 200 so as not to interfere with the attachment of monitoring device 200 to incentive spirometer 100. However, in other embodiments, charging interface 218 may be implemented at or near the base of monitoring device, similar to the embodiments illustrated in FIGS. 2B and 2C, again, so long as they do not interfere with the connection of monitoring device 200 with incentive spirometer 100. For example charging interface 218 may be positioned such that charging interface 218 remain external to air chamber 108 of incentive spirometer 100 (or if there is no impact to the operation of incentive spirometer 100/monitoring device 200, within air chamber 108 of incentive spirometer 100.

In some embodiments, incentive spirometer 100 may have an alternative configuration or air route such that proper usage of incentive spirometer 100 may cause air flow to be reversed, e.g., flow from a top section of incentive spirometer 100 towards a base of incentive spirometer 100, in which case, base 120 may have one or more vents, apertures, or openings. Accordingly, monitoring device 200 may be operatively attached atop incentive spirometer 100 without a need for air openings.

In still other embodiments, as illustrated in FIG. 2E, monitoring device 200 may be implemented or formed as base 120 of incentive spirometer 100. That is, monitoring device 200 need not be a “separate” unit that is attachable to incentive spirometer 100. For example, monitoring device may have one or more charging interface 218 positioned on a bottom surface of base 120/monitoring device 200, although the one or more charging interface 218 may be positioned anywhere that allows rechargeable battery 216 to be charged/recharged. It should be noted that in some embodiments, power to monitoring device/one or more components requiring power may be effectuated through other power sources, such as non-rechargeable batteries, a power supply connectable to a building mains, etc. Other components of monitoring device 200, such as display 212, switches 204/206, etc. may be implemented within or as part of base 120 of incentive spirometer 100.

Further, unlike current approaches to monitoring incentive spirometry, the technology disclosed herein is less complex, allowing medical personnel to more reliably and efficiently ensure compliance by patients with recommended therapy. Current approaches require connection to a laptop or other computer system, complex setups and equipment, and require patients to come to the office. The embodiments of the technology of this disclosure are capable of conducting the required analysis locally at the incentive spirometer without the need for separate equipment, resulting in a less complex system that is smaller and more portable, or alternatively, a unitary system that nevertheless still easy to use. This makes it easier for medical personnel and patients to view the incentive spirometer data at the device, eliminating the need to utilize other equipment. In some embodiments, the monitoring device may include a built in USB connector, enabling the monitoring device to be directly attached to a computer after use to store or review data, or (as discussed above) to charge the monitoring device.

Moreover, the technology of the present disclosure can be taken home by the patient, facilitating better compliance by eliminating the need to go somewhere else to perform the spirometry. Embodiments of the technology may store monitoring data performed at the patient's home or other non-medical personnel environment, which can then be reviewed by the medical personnel at the next meeting. The patient has the capability to reset, reconfigure, and setup the monitoring device at home himself or herself, enabling multiple measurements to be taken and maintained together for review later.

Embodiments of the technology disclosed herein further reduce the need for extensive sterilization techniques. Prior art monitoring solutions allowing for capturing and storing information associated with spirometry require time consuming and/or intensive cleaning and sterilization techniques to enable reuse between patients. Sterilization is required because these prior art solutions are more integrated with the incentive spirometer. Monitoring devices in accordance with the technology disclosed herein, however, reduce the need for such sterilization. The embodiments disclosed are compatible with cheaper, one-patient use incentive spirometers (that are not intended for reuse with another patient and, therefore, no need to sterilize). In this way, the monitoring device can be used in a fast and efficient manner with multiple patients without the need for extensive sterilization techniques. For example, after use with one patient, a monitoring device in accordance with the present disclosure can be removed from the first incentive spirometer, cleaned with a disinfectant wipe, and coupled to a second incentive spirometer for a second patient.

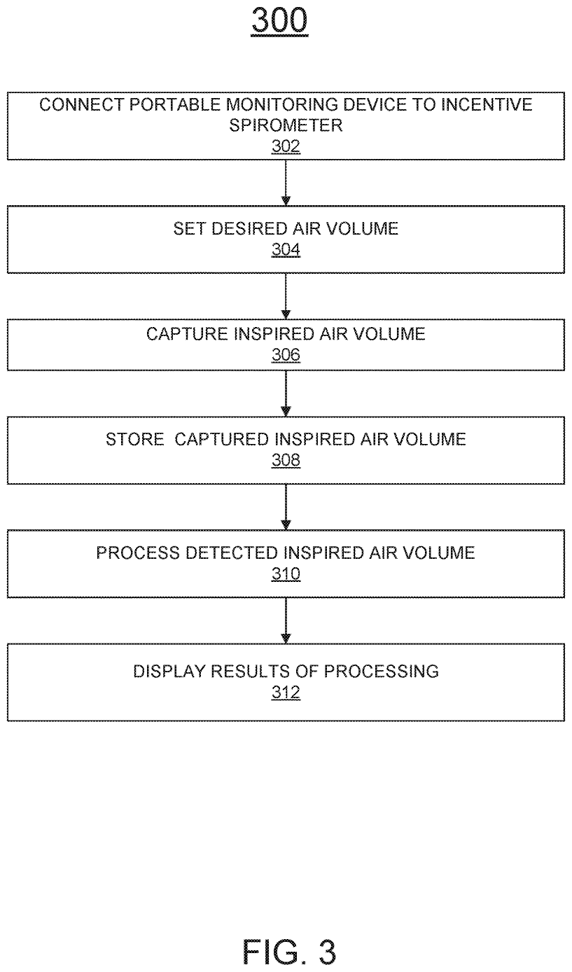

### FIG. 3

FIG. 3 is a flow diagram illustrating an example method in accordance with the technology disclosed. At a high level, method 300 may be performed to monitor incentive spirometry. The operations of the various methods described herein are not necessarily limited to the order described or shown in the figures, and one of skill in the art will appreciate, upon studying the present disclosure, variations of the order of the operations described herein that are within the spirit and scope of the disclosure. Let it be appreciated that operations of method 300 may be performed multiple times.

The operations and sub-operations of method 300 may be carried out, in some cases, by and/or using one or more of the components, elements, devices, and sub-components of monitoring device 200 and/or incentive spirometer 100 (including components thereof as described above), as described with respect to at least FIGS. 1, 2A, 2B, and 4A-7, as well as components, elements, devices, and sub-components, depicted therein and/or described with respect thereto.

In such instances, the description of method 300 may or may not refer to a corresponding component and/or element, but regardless of whether an explicit reference is made, one of skill in the art will recognize, upon studying the present disclosure, when the corresponding component and/or element may be used. Further, it will be appreciated that such references do not necessarily limit the described methods to the particular component and/or element referred to. Thus, it will be appreciated by one of skill in the art that aspects and features described above in connection with (sub-) components, elements, devices, and components, including variations thereof, may be applied to the various operations described in connection with method 300 without departing from the scope of the present disclosure.

Referring now to FIG. 3, aspects of the example method 300 for monitoring incentive spirometry are depicted. The monitoring device is connected to the incentive spirometer at operation 302. At operation 304, medical personnel may set the desired air volume for a patient. The desired air volume may be set as discussed above with respect to FIGS. 1, 2A, and 2B. The desired air volume may correspond to the predicted goal of the user, and may represent a standardized air volume based on a user's sex, weight, height, age, and/or other parameters specific to an individual, including for example, BMI (i.e., body-mass index), exercise history, smoking history, history of cancer, etc. The desired air volume may be an air volume between the range of about 250 ml to about 3000 mL. A person of ordinary skill in the art would know that the desired air volume may vary within the range based on the specifics of the therapy and the capacity of the incentive spirometer.

In various embodiments, the desired air volume is received from the incrementing of, by example, the first switch 204 and/or the decrementing of the second switch 206 discussed with respect to FIG. 2B. For example, the first switch 204 may increment the desired air volume as follows:

$${V}_{ref} = {V}_{ref} + n ( 500 mL ) ,$$

where V_{ref} is the desired volume amount and n is the number of times the first switch 204 is pressed. In the above example equation, the increments are set at 500 mL. This increment is merely for discussion purposes and embodiments of the technology disclosed herein can have increments of various size, depending on the level of specificity desired. The inverse may be used for the second switch 206, where the desired air volume is set as:

$${V}_{ref} = {V}_{ref} - n ( 500 mL ) .$$

Although discussed with respect to first switch 204 and second switch 206, this was merely an example of how the desired air volume may be received. In various embodiments, the desired air volume may be received through a different action of one or more switches of the device, as discussed above with respect to FIG. 2A. A person of ordinary skill would not view this recitation of setting the desired air volume as limiting.

In various embodiments, a display of the monitoring device (such as display 212 of FIG. 2A, for example) may show the desired air volume to the medical personnel to ensure that it is set correctly. In some embodiments, LEDs or other indicators may be included in the monitoring device to indicate whether a desired air volume has been set. For example, a green LED may indicate that the desired air volume is set, while a red or yellow LED may indicate that the desired air volume has not been set yet.

At operation 306, the inspired (or exhaled) air volume of the user is captured. A user inhales through the mouthpiece of the incentive spirometer as during regular incentive spirometry. In various embodiments, the inspired air volume may be captured by one or more sensors of the monitoring device. The one or more sensors may be configured to determine the inspired air volume through various means, including time of flight of the piston within the air chamber as discussed above. In various embodiments, the inspired air volume may be detected through the use of a light signal, a sound signal, or a combination of both.

At operation 308, the monitoring device stores the captured volume of inspired air in a memory or storage component of the monitoring device, like the memory 224 of the monitoring device 200 discussed with respect to FIG. 2A.

At operation 310, the monitoring device analyzes and processes the data obtained by the one or more sensors. In various embodiments, processing an inspired air volume may include measuring an attempt. An attempt may be registered when the piston 110 reaches (i.e., equals) or exceeds an attempt threshold. An attempt threshold could be a specified air volume that may be between about 0 mL to about 1500 ml. For example, if the attempt threshold is 300 ml; an attempt is registered if the piston reaches or exceeds 300 mL. A person of ordinary skill in the art would know that the specified air volume of the threshold may vary within the range based on the specifics of the therapy and the capacity of the incentive spirometer

In embodiments, processing an inspired air volume may include measuring success. A successful attempt may be registered when piston 110 reaches or exceeds the attempt threshold and reaches/exceeds the desired air volume. For example, an attempt threshold may be 250 ml and a desired air volume may be 1500 ml. In this example, a successful attempt will be registered if the piston reaches/exceeds 1500 ml (meaning that the piston had already passed the attempt threshold of 250 ml, registering this as an attempt). It is to be appreciated that more than one attempt may be measured during a session, with a prior attempt being finished when the piston returns to 0 mL and the next attempt beginning when the attempt threshold is reached or exceeded again. In various embodiments, more than one attempt and/or success may be measured over a period of time. In various embodiments, the monitoring device may also record failed attempts, failed attempts occurring when the piston reaches or exceeds the attempt threshold but fails to reach the desired inspired air volume.

In various embodiments, processing an inspired air volume at operation 310 may include measuring compliance. In embodiments, compliance is measured by summating the number of successful attempts over a period of time. For example, the number of successful attempts may be 7 and the time elapsed may be 2 hrs; in this example, compliance will be

$$\frac{7 * {V}_{ref}}{2} ,$$

where V_{ref} is the desired air volume and compliance is measured in mL/hr.

In some embodiments, processing an inspired air volume at operation 310 may include registering a maximum inspired air volume. A maximum inspired air volume (i.e., max volume) refers to highest air volume reached over a period of time. In various embodiments, processing an inspired air volume may include registering a minimum inspired air volume. A minimum inspired air volume (i.e., min volume) refers to the lowest volume reached during a valid attempt (i.e., where the piston has reached or exceeded the attempt threshold) over a period time.

In various embodiments, more than one attempt may be measured over a period of time (t). In embodiments, the period of time (t) may be in units of years, months, weeks, days, hours, minutes, seconds, or any combination thereof.

In some embodiments, processing at operation 310 may include measuring success of an attempted inspiration (an inspired air volume during a period of time). Success (i.e., successful inspiration) may be registered when piston 110 reaches or exceeds the certain threshold and reaches/exceeds the desired air volume. For example, a certain threshold may be 300 ml and a desired air volume may be 1400 ml; a success will be registered if the piston reaches/exceeds 1400 mL. It is to be appreciated that more than one successes may be measured. In various embodiments, more than one success may be measured over a period of time (t). In embodiments, the period of time (t) may be in units of years, months, weeks, days, hours, minutes, seconds, or any combination thereof.

A counter may be incremented when a success is registered during a period of time (t) at operation 310. In various embodiments, the period of time (t) may be in units of years, months, weeks, days, hours, minutes, seconds, or any combination thereof. In some embodiments, operation 310 may include summing the plurality of increments over the period of time (t).

In some embodiments, the following algorithm may be employed:

$${V}_{acc} = \sum {V}_{{i}_{c}} , c ϵ [ 0 - p ] ,$$

where V_{acc} is the accumulated inspired air volume, Vᵢ_{_{c}} is the volume of inspired air following each successful attempt c. A time period (t) may have as many as p successful attempts. Thus, each Vᵢ represents the desired air volume set by the user (V_{ref}) at a given instant c for discrete events. The accumulated inspired air volume can then be compared with the desired air volume V_{ref} to determine whether a patient as achieved a desire air volume over time, or complied with the recommended therapy.

In various embodiments, the monitoring device can measure the inhale/respiratory rate of a patient. The inhale/respiratory rate measured how fast it takes for a single attempt (starting from when the piston reaches the attempt threshold) to reach the desired air volume. For example, the inhale/respiratory rate may be calculated as:

$$R R = \frac{( {V}_{ref} - {V}_{thresold} )}{t} ,$$

where RR is the respiratory rate and V_{threshold} if the attempt threshold. The calculated RR can be used to calculate an average respiratory rate (RR_{avg}) for the patient over a number of successful attempts, for example

$$R {R}_{avg} = \frac{\sum R R}{Δ t} .$$

At operation 312, the monitoring device can display the results of the processing operation. In some embodiments, the results displayed could include, but is not limited to: attempt(s); success(es); compliance; maximum inspired air volume; and minimum inspired air volume; or a combination thereof.

In various embodiments, operation 312 may include displaying an elapsed time. In embodiments, the elapsed time may be in units of years, months, weeks, days, hours, minutes, or seconds. In embodiments, the elapsed time may be displayed by the display 212. In embodiments, the elapsed time may be displayed by the plurality of LEDs 214.

In various embodiments, operation 312 may include displaying a desired air volume. In embodiments, the desired air volume may be displayed by the display 212. In embodiments, the desired air volume may be displayed by the plurality of LEDs 214.

The foregoing description of FIG. 3 was made with reference an inspiratory breathing event during which a user inhales through the mouthpiece of the incentive spirometer as during regular incentive spirometry. However, example method 300 can be applied to expiratory breathing events, during which a user exhales through the mouthpiece. For example, at operation 306 expired air volume of the user can be captured in a manner substantially similar to that described above. Operations 302, 308, and 312 may then proceed as set forth above, but based on the captured expired air volume. Additionally, operation 312 may also include displaying FEV, FVC, PEFR, or a combination thereof, alone or with other metrics described above.

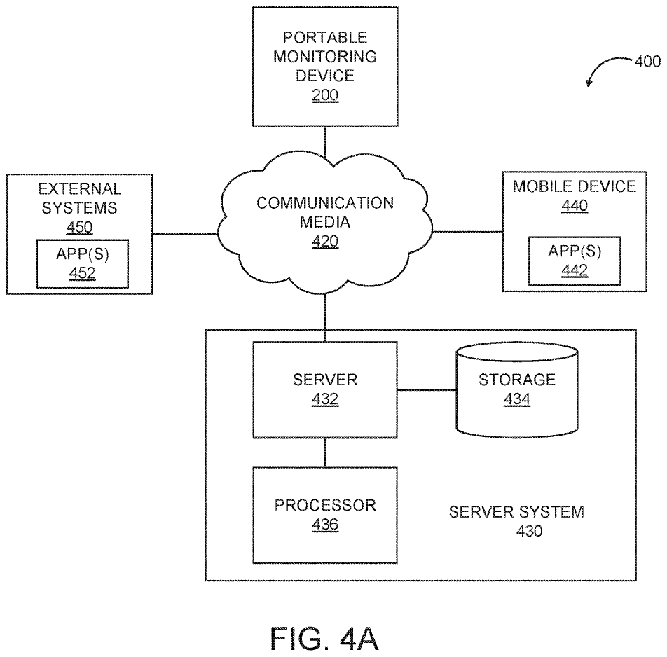

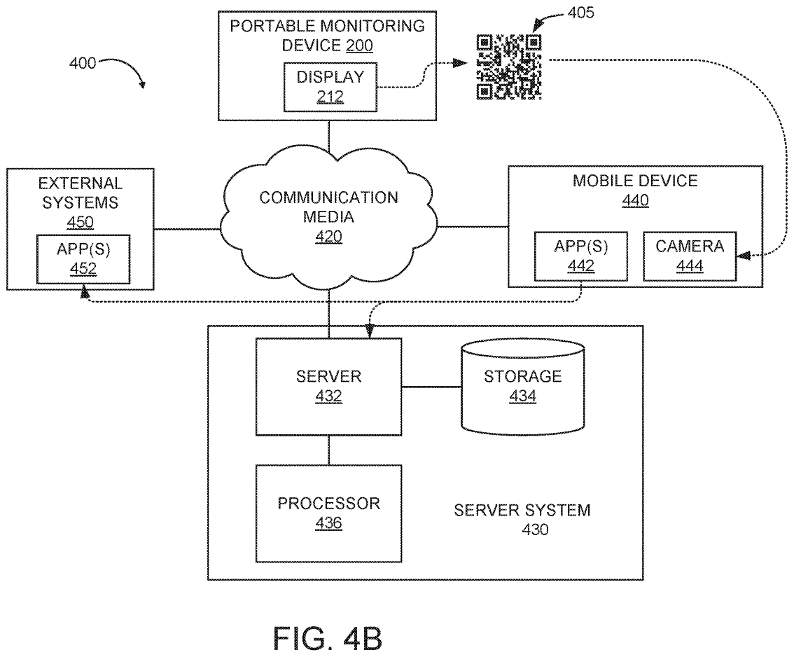

### FIGS. 4A-4B

There may be a need or want to maintain a record of the data and results obtained through the technology discussed herein. In various embodiments, the monitoring device may include an interface to transmit data to a server for storage. FIG. 4A depicts example environment 400, which may be used in connection with implementing embodiments of the disclosed systems, methods, and devices.

As shown in FIG. 4A, environment 400 may include one or more of monitoring devices 200, one or more mobile devices 440, server system 430 and one or more external systems 450. Monitoring device 200 can be coupled to the one or more mobile devices 440, one or more external system 450, and the server system 430 via communication media 420. Monitoring device 200 refers to the monitoring device 200 described above in connection with FIG. 2A-2C and, while not depicted in FIG. 4A, may also include the charging station 220. As will be described in detail herein, monitoring device 200, mobile device 440, external systems 450, and/or server system 430 may exchange communications signals, including information gathered from one or more applications supported by monitoring device 200 (and/or charging station 220), and other aspects of content for display on monitoring device 200 via communication media 420.

Monitoring device 200 may communicate with other devices (e.g., mobile device 440, external systems 450, etc.) and/or with one another over communication media 420 with or without the use of server system 430, for example, through a communication channel that links them together. In various embodiments, monitoring device 200/or server system 430 may be used to perform various processes described herein and/or may be used to execute various operations described herein with regard to one or more disclosed systems and methods. Upon studying the present disclosure, it will be appreciated that environment 400 may include multiple monitoring devices, mobile devices 440, communication media 420, server systems 430, server 432, processors 436, and/or storage 434. Moreover, interested parties (e.g., medical personnel, family members, etc.) may be able to access the server systems 430 to read data and monitor the patient through external systems 450. For example, a medical personnel (not pictured) may be connected to server system 430 in a similar manner as the monitoring device (discussed in greater detail below) using a mobile device 440 or external systems 450, enabling the medical personnel to monitor the patient's performance remotely without the need for the patient and medical personnel to be in the same location. Non-limiting examples of mobile device 440 include: smartphones; tablets; laptops; desktops; PDAs; among other computing devices.

External systems 450 may refer to health care provider or other third-party systems operated by personnel for to monitor the patient's performance remotely. Example health care providers or other third parties include, but are not limited to, hospitals, skilled nursing facilities (SNF), health, health care providers, and the like. These health care providers or other third parties may operate systems that comprise one or more devices, such as smartphones; tablets; laptops; desktops; PDAs; among other computing devices, which make up a system. In some embodiments, external systems 450 may maintain electronic medical records (EMR) or electronic health records (HER) of a plurality of patients that are organized according to a medical record numbers (MRN) or other identifier that uniquely identifies each patient and corresponding EMR or EHR.

The external systems 450 may also maintain other patient monitoring systems and information obtained from these systems can be maintained in the ERM/EHR according to MRNs. As described above, monitoring device 200 may communicate with the external systems 450, which can then integrate the information obtained from monitoring device 200 with information from other monitoring systems to provide holistic medical care to each patient by using information monitoring device 200 to inform on information obtained from the other monitoring systems. Examples of other monitoring systems includes, but are not limited to, continuous glucose monitoring devices; blood pressure monitoring device; heart rate monitoring device; anticoagulation testing devices; electrocardiogramavices; maternity care monitoring devices; medical alert systems; pediatric monitoring devices; pulse oximeters; medication monitoring devices; and smart scales, among others.

Mobile device 440 may comprise one or more applications 442 installed thereon as clients for interfacing with server system 430 and/or monitoring device 200. Information exchanges between monitoring device 200 and other components of environment 400 may be handled, for example, by application 452 implemented as a software module. Application 452 may include an application programming interface (API) which defines the manner in which mobile device 440 may interact with the monitoring device 200 and/or server system 430. One or more applications 442 may be “thin,” in which case processing is primarily carried out server-side by server system 430, such as a browser application, which simply requests, receives, and renders webpages at mobile device 440, while the server system 430 is responsible for generating the webpages and managing functions. Alternatively, the one or more applications 442 may be “thick,” in which case processing is primarily carried out client-side by mobile device 440. It should be understood that mobile device 440 may perform any amount of processing, relative to server system 430, at any point along this spectrum between “thin” and “thick,” depending on the design goals of the particular implementation. External systems 450 may comprise an application 452 that is similar to application 442 and configured manage information exchanges between external systems 450 and monitoring device 200, as well as other components of environment 400.

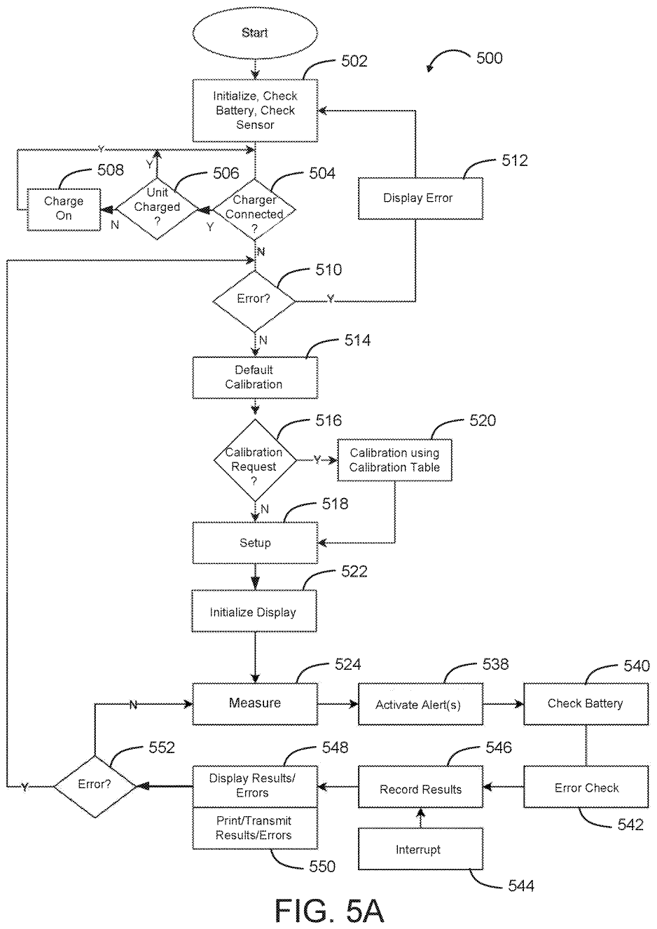

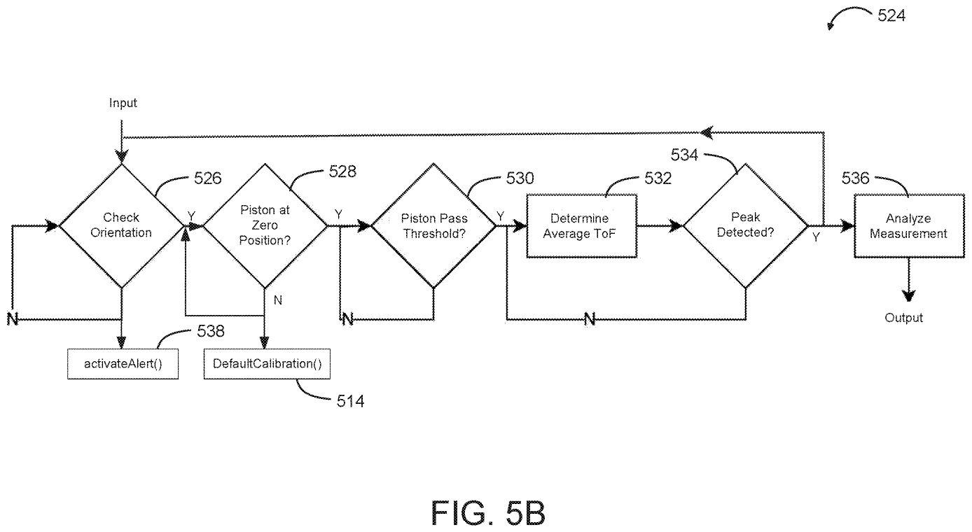

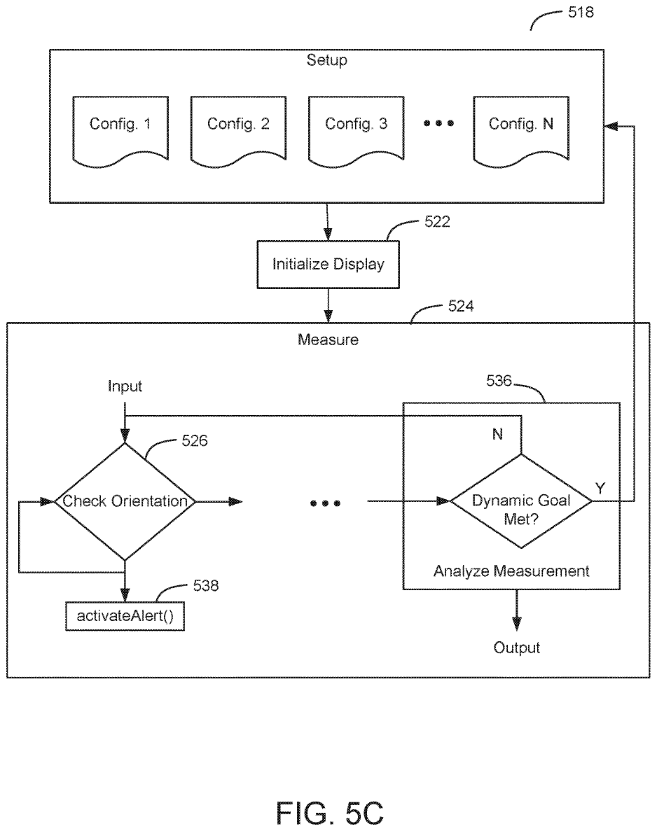

Applications 452 and 442 may be configured to generate alerts and/or notifications that can be presented to a user of the corresponding computing device based on inspiratory/expiratory metrics monitored by the monitoring device 200. For example, metrics may be transmitted to application 452 and/or one or more applications 442 and a notification generated and communicated to external systems 450 or mobile device 440 responsive to the metric meeting a threshold or achievement. Alerts or notifications can be provided as, but not limited to, short message server (SMS) or multimedia messaging service (MMS) text messages, e-mail, phone calls, automated text to speech communications, push notifications on computing devices, and so on. In some examples, alerts or notifications may be presented via display 212 (e.g., visual alerts/notifications), as well as via speakers 222 (e.g., audio alerts/notifications). The alters/notifications may, in part, depend on whether the target recipient is a health care provider or patient. For example, if maximum/minimum inspired/expired inspired air volumes falls below the desired air volume, a notification may be generated on application 452 to inform the health care provider that the patient's deficiency. Similarly, the notification can be generate on application 452 where the patient has failed to use the monitoring device 200 consistency or as required. In the case of one or more applications 442, a notification can be generated to incentives the patient in accordance with gamification techniques described above. As another example, one or more applications 442 may generate notifications to remind the patient to use the monitoring device 200. Example reminders can include fixed and/or adaptive usage reminders, as described below in connection with FIG. 5A.

Application 452 and one or more applications 442 may also be configured to enable remote usage of monitoring device 200. For example, user inputs received by switches 204 and 206 may be received on/through application 452 and/or 442 in various embodiments. In this case, application 452 or 442 may generate a user interface that includes input devices, such as through buttons, graphical icons, mouse, keyboard or other user interaction devices, through which a user may interact to perform the functions of first switch 204 and second switch 206. The application 452 or one or more applications 442 may then communicate the input to monitoring device 200, which can change its operation in a manner described above with connection to first switch 204 and second switch 206.

As mentioned, communication media 420 may be used to connect or communicatively couple monitoring device 200, external systems 450, and/or server system 430 to one another or to a network, and communication media 420 may be implemented in a variety of forms. For example, communication media 420 may include an Internet connection, such as a local area network (LAN), a wide area network (WAN), a fiber optic network, internet over power lines, a hard-wired connection (e.g., a bus), and the like, or any other kind of network connection. Communication media 420 may be implemented using any combination of routers, cables, modems, switches, fiber optics, wires, radio (e.g., microwave/RF links), and the like. Further, communication media 420 may be implemented using various wireless standards, such as Bluetooth, Wi-Fi, 3GPP standards (e.g., 2G GSM/GPRS/EDGE, 3G UMTS/CDMA2000, 4G LTE/LTE-U/LTE-A, 5G). Upon reading the present disclosure, one of skill in the art will recognize other ways to implement communication media 420 for communications purposes.

Likewise, though not shown, it will be appreciated that a similar communication medium may be used to connect or communicatively couple server 432, processors 436, and/or storage 434 to one another, in addition to other elements of environment 400. In example embodiments, communication media 420 may be, or include, a wired or wireless wide area network (e.g., cellular, fiber, and/or circuit-switched connection) for monitoring device 200 and/or server system 430, which may be relatively geographically disparate; and in some cases, aspects of communication media 420 may involve a wired or wireless local area network (e.g., Wi-Fi, Bluetooth, unlicensed wireless connection, USB, HDMI, and/or standard AV), which may be used to communicatively couple aspects of environment 400 that may be relatively close, geographically. In various embodiments, server system 430 may be co-located with the monitoring device (e.g., in the same office, etc.), while in other embodiments the server system 430 may be remotely located (e.g., a data center, cloud system, etc.).

Server system 430 may provide, receive, collect, or monitor information from the monitoring device 200, such as, for example, attempts, successes, compliance, elapsed time, desired air volume, minimum inspired air volume, maximum inspired air volume, FEV, FCV, PEFR, and the like. Server system 430 may be configured to receive or send such information via communication media 420. This information may be stored in storage 434 and may be processed using processors 436. In some embodiments, some information may be removed from the information gathered, for example, metadata, envelopes, IP addresses, personally identifying information and/or other information. For example, processors 436 may include an analytics engine capable of performing analytics on information that server system 430 has collected, received, or otherwise interacted with, from the monitoring device 200. In embodiments, server 432, storage 434, and processors 436 may be implemented as a distributed computing network or as a relational database or the like.

Server 432 may include, for example, an Internet server, a router, a desktop or laptop computer, a smartphone, a tablet, a processor, a component, or the like, and may be implemented in various forms, including, for example, an integrated circuit or collection thereof, a printed circuit board or collection thereof, or in a discrete housing/package/rack or multiple of the same. Server 432, according to various embodiments, may be a Systems and Organization Controls (SOC) 2 compliant server. The SOC specifies how organizations should manage customer data to provide security, availability, processing integrity, confidentiality, and privacy to data processed and maintained on server system 430.

In embodiments, server 432 directs communications for monitoring device 200 over communication media 420. Server 432 may update information stored on monitoring device 200. Server 432 may send/receive information to/from the monitoring device 200 in real time or sporadically. Further, server 432 may implement cloud computing capabilities for the monitoring device 200.

In embodiments, storage 434 stores a respiratory database of patients. The respiratory database may store information from the monitoring device 200, such as, for example, attempts, successes, compliance, elapsed time, desired air volume, minimum inspired air volume, maximum inspired air volume, FEV, FCV, PEFR, and the like. The information may be stored in association with a unique identifier corresponding to each patient. In some cases, the unique identifier may be a MRN, but other identifiers may be used. The respiratory database may also maintain a history of medical problems for each patient. The respiratory database may be accessible to external systems 450 through authentication (e.g., credential verifications such as username/password, multi-factor authentication, or any other authentication technique) of personnel. Based on a request for a particular patient from an external system 450, server 432 may retrieve information about the particular patient from storage 434 and transmit the information to application 452 running on external systems 450.

In embodiments, server 432 may comprise artificial intelligence for determining risk stratification from information held in storage 434. Risk stratification, as used herein, refers to a process of assigning a health risk status to a patient, and using the patient's risk status to direct and improve care. The artificial intelligence may comprise a machine learning algorithm trained on historical data comprising inspiratory/expiratory metrics and health risks (e.g., probability that given inspiratory/expiratory metrics correspond to a given medical condition) to assign a health risk status to a patient based on information received from monitoring device 200. In operation, storage 434 may receive current inspiratory/expiratory metrics from monitoring device 200, either directly from monitoring device 200 or via one or more applications 442 or application 452, for a set period of time up to a current time. The current inspiratory/expiratory metrics can be applied to the trained machine learning algorithm, which calculates a probability for a plurality of health risks to determine a risk stratification by assigning a health risk status to the patient according to calculates probabilities. The risk stratification can then be communicated to external systems 450 or mobile device 440 for use in directing and improving health care provided to the patient.

In embodiments, machine-readable image identifiers can be used for accessing and managing information collected by monitoring device 200. Machine-readable image identifiers refers to an image that contains information specific to an item or patient associated to the image. An illustrative example of a machine-readable image identifier is a quick-response (QR) code. Other examples may be machine readable barcodes or other images that uniquely identify an item or patient.

FIG. 4B depicts an example of environment 400 utilizing a QR code, as an illustrative example, for managing and accessing information collected by monitoring device 200. FIG. 4B is substantially similar to FIG. 4A, except that environment 400 also includes a camera 444 or other image capture device. Also depicted in FIG. 4B is the display 212 of monitoring device 200, which in this example can display a QR code 405. The QR code may contain information that uniquely identifies a patient, such that mobile device 440 can scan (or capture an image thereof) QR code 405 using camera 444. The scan (or image) of QR code 405 is provided to one or more applications 442, which decodes the QR code 405 to obtain the unique identifier of the patient. The identifier can be supplied to external systems 450 and/or server 432 for retrieving information about the patient, such as historical inspiratory/expiratory metrics, EMR/EHR, etc. The retrieved information can then be displayed on mobile device 440 or forward to another device, such as external systems 450.

As an example, display 212 may display information for usage of monitoring device 200 over a first time period (e.g., current measurements, measurements over the most recent 12 hours, etc.) and QR code 405. Mobile device 440 then uses camera 444 to obtain the identifier of the patient and retrieve information of the patient stored in storage 434. For example, the identifier of the patient can used to access respiratory database and obtain any information contained therein for the identified patient. The obtained information can then be displayed on mobile device 440, which may include historical information (e.g., past 3 days, past week, all data, etc.). As another example, QR code can be used to commit recent information collected by monitoring device 200 to storage 434 through obtaining the patient identifier and using the identifier to store the recent information to the respiratory database.

### FIGS. 5A-5B

FIG. 5A is a flow diagram illustrating another example method in accordance with the technology disclosed. At a high level, method 500 may be performed to monitor incentive spirometry, for example, to monitor for compliance with using an incentive spirometry in accordance with the embodiments disclosed herein. The operations of the various methods described herein are not necessarily limited to the order described or shown in the figures, and one of skill in the art will appreciate, upon studying the present disclosure, variations of the order of the operations described herein that are within the spirit and scope of the disclosure. Let it be appreciated that operations of method 500 may be performed multiple times.

The operations and sub-operations of method 500 may be carried out, in some cases, by and/or using one or more of the components, elements, devices, and sub-components of monitoring device 200 and/or incentive spirometer 100 (including components thereof as described above), as described with respect to at least FIGS. 1, 2A, 2B, and 3-4B, and 6, as well as components, elements, devices, and sub-components, depicted therein and/or described with respect thereto.

In such instances, the description of method 500 may or may not refer to a corresponding component and/or element, but regardless of whether an explicit reference is made, one of skill in the art will recognize, upon studying the present disclosure, when the corresponding component and/or element may be used. Further, it will be appreciated that such references do not necessarily limit the described methods to the particular component and/or element referred to. Thus, it will be appreciated by one of skill in the art that aspects and features described above in connection with (sub-) components, elements, devices, and components, including variations thereof, may be applied to the various operations described in connection with method 300 without departing from the scope of the present disclosure.

Referring now to FIG. 5A, aspects of the example method 500 for monitoring incentive spirometry are depicted. The monitoring device can be connected to an incentive spirometer as described herein. To start the method 500, the monitoring device is turned on and executes an initialization function during which power is supplied to the monitoring device and components therein, at operation 502. Operation 502 may include checking if an incentive spirometer is coupled to the monitoring device, for example, as described above in connection with FIGS. 2A-2D.

Operation 502 may also initialize a check battery operation, and a check sensor operation can be executed. The check battery operation can include operation 504, which determines whether or not a charging station (e.g., charging station 220) is connected. If one is connected, method 500 proceeds to operation 506, where method 500 determines whether or not the monitoring device is charged (e.g., rechargeable battery 216 is charged). If the monitoring device is not charged, method 500 proceeds to operation 508 where the charging station is implemented/enabled to charge the monitoring device.

If the monitoring device is charged, or the charging station is not coupled to the monitoring device, method 500 proceeds to operation 510. At operation 510, method 500 determines if an initialization error has occurred. Example errors can include, but are not limited to, the rechargeable battery has a charge below a set threshold charge, an incentive spirometer is not detected as being connected to the monitoring device, the incentive spirometer is improperly orientated (e.g., upside down or other orientation that inhibits operation of a connected incentive spirometer), a sensor is malfunctioning or not present (e.g., IMS sensors 226 and/or sensor 208 are malfunctioning or not present), to name a few. Orientation of the incentive spirometer and/or monitoring device may be based on IMU sensors that detect orientation. It should be understood that other error conditions can be checked for in the event that the initialization operation at 502 cannot commence.

If an error is detected at operation 510, method 500 proceeds to operation 512, where an error display function is triggered that presents an error code or other identifier representative of the detected error to a user. For example, the error code may be displayed on display 212. As another example, the error code may be communicated to a remote system (e.g., external systems 450 or server system 430) for storage and/or presentation to health care providers. Once the error is presented (at 542), method 500 may return to operations 502 for re-initialization.

If an error is not detected at operation 510, method 500 proceeds to operation 514 where a default calibration function is executed. Default calibration can refer to an initial comparison and resetting of the state/position of a piston, e.g., piston 110. That is, a current state/position of the piston can be compared to a set default “resting” or initial/reset state that may be set in memory. The default resting state can be a position or location of the piston relative to the air chamber, e.g., air chamber 108, such as at/near a “0” volume position/mark. If the current state/position of the piston comports with its default resting state/position, calibration can be considered complete. If, for example, the position of the piston is not its default resting state/position, an error or instruction can be presented informing the user or medical personnel to move the piston to its default resting state/position. For example, a patient may have inverted the incentive spirometer and upon righting the incentive spirometer, the piston may become lodged or stuck. As another example, the patient may be holding the incentive spirometer in such a way, e.g., angled orientation, that the piston is not at its default resting state/position. An error or instruction to reset the piston may be presented and a user/medical personnel may, e.g., manually reset the piston by shaking the incentive spirometer to loosen the piston, inspire or exhale through the incentive spirometer to move/dislodge the piston, and so on. It should be noted that default calibration 514 can be performed after initialization or power up, or can be performed after each time the piston moves pursuant to user inhalation or expiration.

Operation 516 detects a calibration request and either triggers operation 520 or proceeds to operation 518, where a setup function is executed. Operation 518 may be performed to “setup” or configure the monitoring device, e.g., alarm settings, max/min volumes, target volumes, etc., i.e., configuration or setup parameters according to which the monitoring device operates for different users, different periods of use, and so on. Operation 518 may also comprise configuring the monitoring device with default or standard operating parameters that can be used or can be changed through actuation of the aforementioned switches, for example.

Operation 520 is performed because the monitoring device can be used with a variety of different incentive spirometers, where one incentive spirometer may have an air chamber with a different diameter than another incentive spirometer. Thus, the measured values/volumes by the monitoring device may differ even though the amount of, e.g., vertical displacement/time of flight measurement of the piston when in use may be the same. These parameters or calibration values may be represented as numerical values that can be input into a lookup table, e.g., calTable(calValue[i]), e.g., a table with a first column comprising sensor measurement values (whether vertical displacement, time of flight, and so on), and a second column that maps or equates such sensor measurement values with an air volume. In this way, when a user inspires or expires air through the incentive spirometer, and the monitoring device senses and determines an inspired/expired air volume, that inspired/expired air volume value can be checked against the table value to equate the sensed air volume value with a measurement. In operation, when the user inspires/expires air through the incentive spirometer, the monitoring device can determine the amount that the piston has been displaced from its initial or default resting state. The calibration table can be checked to find that amount of displacement, which in the calibration table, is mapped to a particular air volume. Thus, the sensed piston displacement can be equated to an air volume measurement associated with that inspiration/expiration event.

Accordingly, different calibration tables may be configured or set for different incentive spirometers. The calibration request detected at operation 516 can be detected pursuant to a user manually requesting or initiating operation, or may be automatically triggered upon sensing that the monitoring device has been connected to an incentive spirometer. Moreover, monitoring device may comprise sensors (such as those discussed above) or other sensors that can detect a diameter of the air chamber of an incentive spirometer, and an appropriate calibration table can be accessed or enabled for use.

Once the monitoring device is setup at operation 518, a display can be initialized at operation 522. Display initialization may comprise configuring the display to present desired/required information. As discussed above, different patients may have different treatment plans, and thus, different information should be presented on the display. As another example, different medical personnel, depending on the use case scenario (or again, depending on the patient) may wish to see or view different information or metrics.

At operation 524, a measure function is triggered that measures inspiratory/expiratory metrics and outputs the metrics. Operation 524 can utilize sensors of the monitoring device to measure metrics including, but are not limited to: number of attempts; whether a patient succeeded per the parameters; compliance; maximum inspired air volume (i.e., max volume); minimum inspired air volume (i.e., min volume); acceleration of the piston; total time air input was within recommended levels; percentage of time spent within the acceptable ranges for a success; FEV; FVC; PEFR; among others, as described above in connection with FIG. 2A.

In some examples, operation 524 may include detecting an inspired air volume, as described above in connection with FIG. 3. For example, operation 524 may include capturing inspired air volume, as described above in connection with operation 306. The captured volume of inspired air can be stored, as described above in connection with operation 308, and processed/analyzed, as described above in connection with operation 310. Further details are provided above in connection with FIG. 3.

Another example implementation of a measure function that can be executed at operation 524 is depicts in FIG. 5B. As shown in FIG. 5B, an input is received as a breathing event. For example, a user inhales through the mouthpiece of the incentive spirometer. In various embodiments, the inspired air volume may be captured by one or more sensors of the monitoring device.

Upon receiving a breathing event, the orientation of the incentive spirometer is checked at operation 526. The IMU sensors of a connected monitoring device may be configured to determine an orientation of the incentive spirometer. Operation 526 may determine an orientation of the incentive spirometer based on data from the IMU sensors and determine if the orientation satisfies a preset orientation criteria. For example, the preset orientation criteria may define a maximum acceptable tilt from perpendicular to the ground (e.g., tilt from a upright orientation position). In an example, the maximum acceptable tilt may be 85 degrees in X-, Y- or Z-directions relative to perpendicular from ground. Other maximum acceptable tilts may be utilized according to a desired implementation. If the orientation exceeds the maximum acceptable tilt (e.g., “N” at operation 526), operation 524 repeats. In some examples, an alert may be triggered (e.g., operation 538) to signify to a user or medical care personnel that the incentive spirometer is not orientated properly.

If operation 526 determines in the affirmative, operation 524 can proceed to operation 528 where a position of the piston (e.g., piston 110) is checked to confirm that the piston is located at a zero (or resting) position of the incentive spirometer (e.g., as depicted in FIG. 1). In some examples, the zero position, pistonZero( ) of the piston may represent a starting position of the piston for a next breathing event, as well as an ending position of the piston for a current breathing event. If the piston is not at the zero position, the operation proceeds to operation 514 to repeat calibration. As described above, a resting position may be equated with a particular height or level of the piston within the air chamber, and a calibration table can be checked to determine if the displacement of the piston equates to a zero or resting position. It should be understood that the displacement of the piston may not always be exact to some nth degree, e.g., there may be some allowable variance or tolerance. Additionally, a piston may have some height/width, where the top of the piston may be equated with a zero or resting position, and where the zero or resting position corresponds to some default height within the air chamber, e.g., a couple of centimeters. Ultimately, the zero position of the piston can be checked to ensure accurate measurements/proper use of the incentive spirometer, where, e.g., the piston should be at its zero or resting position before an inspiration/expiration attempt can be initiated. This can prevent successful attempts being recorded when a user or patient, e.g., makes multiple “short” inspirations that result in meeting a target air volume, instead of first and “correctly” letting the piston go to its resting position before an inspiration attempt.

When operation 528 is affirmative, operation 524 proceeds to operation 530 to register an attempt. For example, the inspired air at the input causes the piston to raise, and an attempt can be detected where the piston is raised above a threshold (e.g., 250 ml is some examples). If the piston is raised above the threshold, the breathing event is registered and counted as an attempt and the operation 524 proceeds to operation 532. Otherwise, operation 524 repeats operation 530 until an attempted is detected.

At operation 532, a time of flight is tracked through a number of breathing events. Operation 532 may determine a time of flight for each breathing event and determine an average time of light from a number of breathing events, including a current breathing event. In some cases, the number of breathing events must meet a minimum number before the average time of flight is calculated so to provide a sample size where the determined average can represent a true average for the user. In an illustrative example, the minimum number of breathing events is 10, but other minimums may be implemented as desired for a given application. In some examples, operation 532 utilizes a sliding window average that computes an average time of flight for a set number of the most recent breathing events (e.g., the 10 most recent breathing events). The time of flight can be tracked through, for example, sensors 208, as described above in connection with FIG. 2A.

At operation 534, a peak piston position is detected, for example, through sensors 208. If a peak is not detected, operation 524 returns to operation 532 for a next breathing event. As an example, for a first number of breathing events that is less than the minimum number, operation 532 determines a time of flight for each breathing event, and operation 534 records the height that the piston is raised for that breathing event. For a next breathing event (e.g., the minimum number of breathing events), operation 532 determines a time of flight for the breathing event, and determines an average time of flight for the collective breathing events. Operation 534 also records the height of the piston, and determines if this height is a peak position of the piston relative to the preceding breathing events. If the height of the piston for the instant breathing event is not a peak, operations 532 and 534 are repeated for a next breathing event.

Once a peak is detected at operation 534, operation 524 repeats for a next breathing event. A new measurement may not be performed until the piston returns to the zero (or resting) position, described above in connection with operation 528.

Additionally, once the peak is detected operation 536 analyzes and processes the measurements to generate an output of results. For example, at operation 536, the peak height of the piston is converted to a peak in terms of volume (e.g., mL) and calculates a maximum inspired air volume based on the average time of flight and the peak volume. Additionally, operation 536 may increment a count of attempts based on a number of breathing events that passed operation 530. Operation 536 can also detect a successful attempt in a case where the peak volume exceeds a desired air volume input by medical personnel and increment a count of successful attempts according to the detected successful attempts.

Operation 536 may include determining an inhalation rate from the number of breathing events. Inhalation rate refers to a volume of inspired air over a defined timeframe. A timeframe may be set and operation 536 may compute a total volume of air inspired over the time frame. The time frame may be provided in terms of minutes (e.g., 1, 2, 3, etc.), seconds (e.g., 10, 20, 30, etc.), hours, or any desired time frame). Operation 536 may sum the volume of air inspired over that time frame to provide an inhalation rate.

Operation 536 may also determine a maximum tidal volume of a user. For example, over the number of breathing events, the volume of air inspired can be determined for each breathing event by tracking the piston position and converting the position to a volume of inspired air, as described herein. The volume of air inspired for each breathing event may be considered a tidal volume for that breathing event. Operation 536 may analyze the tidal volumes for the number of breathing events and determine a maximum tidal volume for a single inhalation by identifying a maximum tidal volume for the number of tidal volumes.

This maximum tidal volume represents an amount of air that a person can inhale in a single breath. It is a measure of the strength of the respiratory muscles and the size of the lung. Measuring the maximum tidal within a single inhale cam be important because it can help to assess lung function and track progress over time. Measuring maximum inhalation volume can be helpful for people with respiratory problems such as asthma, chronic obstructive pulmonary disease (COPD), and cystic fibrosis. These conditions can weaken the respiratory muscles and make it difficult to inhale deeply. By measuring maximum inhalation volume, medical care providers can track how these conditions are affecting lung function and adjust treatment plans as needed. In addition, measuring maximum tidal can be helpful for people who have undergone surgery or other procedures that can affect lung function.

Referring back to FIG. 5A, outputs of the results of operation 534 are provided to operation 538. The output may include, but not limited to: count of attempt(s); count of success(es); compliance; maximum inspired air volume; minimum inspired air volume; acceleration of the piston; total time air input was within recommended levels; percentage of time spent within the acceptable ranges for a success; FEV; FVC; PEFR; inhalation rate; maximum tidal for a single inhalation, and combinations thereof.

Operation 538 executes an alert activation function based the results output by operation 524. In some examples, alert activation function may trigger an alert (e.g., auditory and/or visual alerts) to a user indicative of a detected attempt; a detected success attempt (e.g., the air chamber 108 reaches or exceeds the threshold and desired air volume input by medical personnel); a detected unsuccessful attempt (e.g., the air chamber 108 does not reaches or exceeds the threshold, but not the desired air volume input by medical personnel); and combinations thereof. For example, a first alert may be triggered when an attempt is detected and a second alter triggered when a success is detected or a third alert when the attempt is unsuccessful. Alerts may be emitted by a speaker (e.g., speakers 222), for example, as a buzzer or other sound to alert the user of the result. As another example, alerts may be displayed by LED (e.g., LEDs 214), which may emit light according to the result. Different sounds, colors, or combinations thereof can be presented to signify different result. For example, a buzzer sound may be emitted to signify that an attempt was detected, a green LED may signify a successful attempt, and a red LED may signify an unsuccessful attempt. In embodiments, different combinations of colors/sounds may be used to signify different combinations of achievements and results.

Furthermore, operation 538 may generate an alert to signify to a user or medical care personnel that the incentive spirometer is not being used in compliance. For example, an alert (audio or visual) may be generated to signify improper orientation (e.g., as detected in operation 526 of FIG. 5B) and/or the piston is not at the zero position (e.g., as detected in operation 528 of FIG. 5B).

Further still, operation 538 may generate usage reminders to remind the user to use the incentive spirometer. The frequency at which an incentive spirometer should be used depends on the individual's medical condition and the reason for using the device. An illustrative example, the incentive spirometer may be used 10-15 times per hour while the user is awake, which can be done in sets of 5-10 breaths, with a short rest in between sets. Reminders can be provided as fixed usage reminders and/or adaptive usage reminders. A fixed usage reminder reminds a user to use the incentive spirometer at least once every fixed time interval (e.g., once every 5 minutes for example). An adaptive usage reminder can remind a user to use the incentive spirometer a fixed number of times within an hour. For example, assume a prescribed usage of 15 times per hour. Then the average usage will be one use every four minutes. If the user uses the incentive spirometer five times within the first ten minutes, the adaptive usage reminder can be set to 50 minutes divided by 15 minus five (e.g., 50/(15-5)), which is 5 min. Next, if the user uses the incentive spirometer five times within the second ten minutes, adaptive usage reminder can be set to 40 minutes divided by 15 minus ten, which is 8 minutes, and so on. In either case, operation 538 may trigger an alert (e.g., audio and/or visual alert) to signify a fixed or adaptive usage reminder.

In some embodiments, operation 538 may generate an inhalation rate reminder alert that signifies that the inhalation rate of the user is not constant and should be adjusted. For example, an alert may be triggered that notifies the user or medical care personnel that the inhalation rate has deviated from a defined inhalation rate. The defined inhalation rate may be based on an average inhalation rate or a target inhalation rate defined by a medical care provider. An average inhalation rate can be determined according to a sliding window average over a number of timeframes. A instant inhalation rate may be considered to deviate from the defined inhalation when the instant inhalation rate exceeds a maximum threshold deviation or is less than a minimum threshold deviation from the defined inhalation rate. Thus, if an instant inhalation rate is outside of the threshold deviations, an alert can be triggered to notify a user to adjust the user's inhalation rate, thereby training the user to maintain a constant inhalation rate.

Maintaining a constant inhalation rate when using an incentive spirometer can be important because it helps to ensure that the alveoli in the lungs are inflated. If the inhalation rate is too fast, some of the alveoli may not be fully inflated, which can lead to respiratory problems. In addition, a constant inhalation rate helps to prevent the patient from hyperventilating. Hyperventilation is a condition in which the patient breathes too quickly and too deeply, which can lead to dizziness, lightheadedness, and other symptoms. By tracking an inhalation rate relative to a defined inhalation rate, embodiments disclosed herein can help to ensure that users are taking deep breaths that will effectively inflate the alveoli and prevent respiratory problems.

Maintaining a constant inhalation rate can provide for various benefits. For example, embodiments disclosed herein can help to improve lung function by increasing the volume of air that the lungs can hold. This can be helpful for people with respiratory problems such as asthma, COPD, and cystic fibrosis. Additionally, embodiments disclosed herein can help to prevent atelectasis, a condition in which the alveoli collapse, by keeping the alveoli inflated. Furthermore, embodiments disclosed herein can help to increase comfort by reducing shortness of breath and improving oxygen levels in the blood.

At operation 540, a check battery function can be executed. The check battery function may be similar to operations 504-508, where a charge of the rechargeable battery is checked to confirm the charge is above the set threshold charge. Check battery function analyzes current charge of the rechargeable battery to ensure that the monitoring device has sufficient charge to perform subsequent measurements via operation 524.

At operation 542, an error checking function can be executed in order to report any error found at operation 510. Reporting an error can be effectuated through presentation on the display at 548.

At operation 546, the results of the measurement function (e.g., operation 524) can be recorded to memory via a record input function.

In some embodiments, operation 546 may be triggered responsive to an interrupt function triggered at operation 544. In one example, the operation 544 may be triggered to interrupt a sequence of measurements performed by operation 524 and cause the results output from operation 524 to be recorded to memory at operation 546. As another example, a user may have turned the monitoring device, on without having yet connected the monitoring device to the incentive spirometer. To forgo receipt of error notifications at operation 510/512, a user may actuate a button(s)/switch(es) to trigger an interrupt, so that the monitoring device can instead attempt initialization/check the battery/check the sensor(s) operations 502.

In an example implementation, the monitoring device may include a user input device, such as a button or icon on a touch display, that a user may interact with to request interruption. User interaction with the input device may trigger operation 544, which can trigger operation 546 to record the results from operation 524 to memory. The user input device may also include voice commands through speech recognition techniques. In another example, voice commands may be presented to a virtual assistant via a communications circuitry (e.g., communications circuitry 209) and the virtual assistant may trigger operation 544.

The results may also be displayed at operation 548 for presentation to a user or medical care personnel. In some embodiments, the results displayed could include, but is not limited to: count of attempt(s); count of success(es); compliance; maximum inspired air volume; and minimum inspired air volume; total time air input was within recommended levels; percentage of time spent within the acceptable ranges for a success; FEV; FVC; PEFR; inhalation rate; maximum tidal volume of a breathing event; or a combination thereof. Operation 548 may also be triggered to display notifications signifying alerts generated by operation 538, such as textual information describing the generated alert.

The results of operation 524, and any errors detected at operation 542, can also be printed or transmitted to an external system (e.g., external system 450 and/or server system 430) at operation 550. A print out can be generated by a physical printer communicatively coupled to the monitoring device and/or to a computing system that is communicatively coupled to the monitoring device. In another example, a printout may refer to a digital print out, such as a digital document that can be viewed by a user or medical care provider via a computing system. Operation 550 may also commit the results and detected errors (if any) to a storage device (e.g., storage 434) for inclusion in a respiratory database stored therein, as described above in connection with FIGS. 4A and 4B.

At operation 552, up the monitoring device with various configurations according to which dynamic targeting or coaching through setting desired threshold volumes and successful attempts. As illustrated in FIG. 5C, a plurality of such configurations may be input (configuration 1, configuration 2, configuration 3, . . . configuration N). Each configuration may comprise a target inspiration (or expiration) volume and a number of successful attempts that a user must accomplish before a subsequent/next configuration is enabled, where the subsequent/next configuration sets forth parameters for a new/next goal or target.

In operation, a first target or goal set forth in a first configuration may be enabled. As described above, once the monitoring device is setup at operation 518 (in this example, with configuration 1), the display can be initialized at operation 522. Display initialization, as also described above, may comprise configuring the display to present desired/required information. In this example, the display may be initialized to display the target goal set forth vis-à-vis configuration 1.

At operation 524, a measure function is triggered that measures inspiratory/expiratory metrics and outputs the metrics. As discussed above, operation 524 can utilize sensors of the monitoring device to measure metrics including, but are not limited to: number of attempts; whether a patient succeeded per the parameters; compliance; maximum inspired air volume (i.e., max volume); minimum inspired air volume (i.e., min volume); acceleration of the piston; total time air input was within recommended levels; percentage of time spent within the acceptable ranges for a success; FEV; FVC; PEFR; among others, as described above in connection with FIG. 2A.

In some examples, operation 524 may include detecting an inspired air volume, as described above in connection with FIG. 3. For example, operation 524 may include capturing inspired air volume, as described above in connection with operation 306. The captured volume of inspired air can be stored, as described above in connection with operation 308, and processed/analyzed, as described above in connection with operation 310. Further details are provided above in connection with FIG. 3.

As shown in FIG. 5C, similar to FIG. 5B, an input is received as a breathing event. Upon receiving a breathing event, the orientation of the incentive spirometer is checked at operation 526. If orientation is “good,” operation 524 can proceed to operation 528 where a position of the piston (e.g., piston 110) is checked to confirm that the piston is located at a zero (or resting) position of the incentive spirometer (e.g., as depicted in FIG. 1). When operation 528 is affirmative, operation 524 proceeds to operation 530 to register an attempt. For example, the inspired air at the input causes the piston to raise, and an attempt can be detected where the piston is raised above a threshold (e.g., 250 ml is some examples). If the piston is raised above the threshold, the breathing event is registered and counted as an attempt and the operation 524 proceeds to operation 532. Otherwise, operation 524 repeats operation 530 until an attempted is detected. At operation 532, a time of flight is tracked through a number of breathing events. At operation 534, a peak piston position is detected, for example, through sensors 208. If a peak is not detected, operation 524 returns to operation 532 for a next breathing event. Once a peak is detected at operation 534, operation 524 repeats for a next breathing event. Additionally, once the peak is detected, operation 536 analyzes and processes the measurements to generate an output of results.

In this example, the operation 536 analysis/processing may comprise performing a check to determine whether or not a dynamic goal is met. As discussed above, different configurations, e.g., configurations 1, 2, 3 . . . . N, may represent successive, different goals that make up the dynamic goal. For example, if configuration 1 comprises an initial goal of 500 ml inspiration volume at least 3 times, operation 536 may analyze breathing events to determine whether or not this initial goal has been met. As noted above, dynamic targeting/coaching may comprise various parameters/varied execution. That is, the initial goal of 500 ml inspired volume at least 3 times may be configured as successive successful attempts, whereas in other examples, the number of successful attempts at the desired inspired volume goal may be monitored for occurrence over a given period of time rather than successively, and so on. If a dynamic goal is met at operation 536, the process may return to operation 518, where the next configuration, in this example, configuration 2 is enabled. That is, new parameters representative of the next goal or target, e.g., inspired volume of 750 ml at least 3 times, may be used to configure the monitoring device. The display may be initialized at operation 522 to display the new goal, and measuring operation 524 may be repeated until at operation 536, a determination can be made as to whether or not the dynamic goal (now 750 ml inspired volume for three times) has been met. If not, operation may return to the beginning operation 526 of measurement operation 524, and a new breathing event may be measured as described above. This can continue until the current goal has been met, and operation can return to setup operation 518 from measurement analysis operation 536.

It should be understood that any one or more parameters can be set to effectuate dynamic targeting/coaching, not necessarily volume and number of attempts. For example, in some scenarios, it may be desirable to further configure the monitoring device to monitor for a given number of successful attempts in a given time period, where a new target or goal is enabled only after a patient is able to perform a threshold number of successful attempts during some given time period.

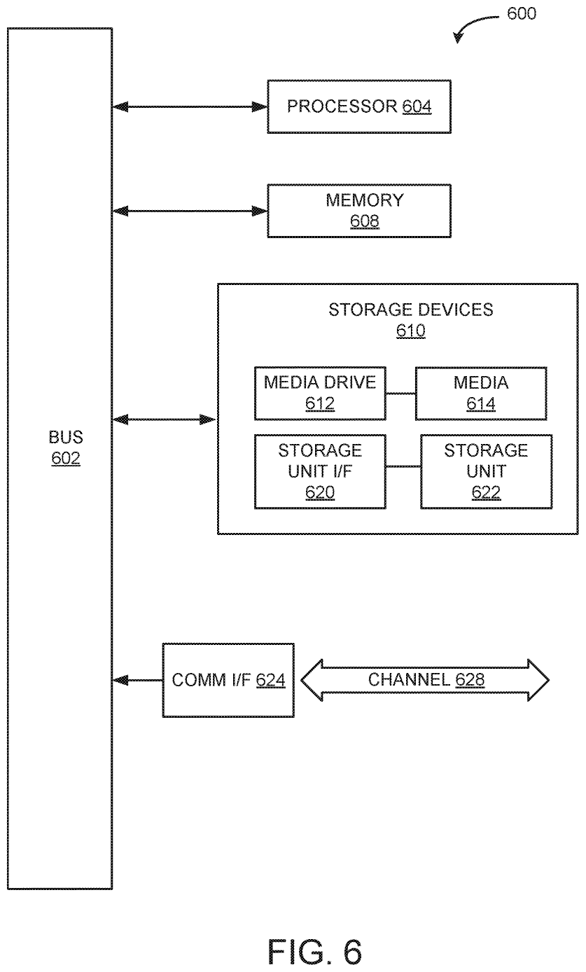

### FIG. 6

FIG. 6 illustrates example computing component 600, which may in some instances include a processor/controller resident on a computer system (e.g., monitoring device 200). Computing component 600 may be used to implement various features and/or functionality of embodiments of the systems, devices, and methods disclosed herein. With regard to the above-described embodiments set forth herein in the context of systems, devices, and methods described with reference to FIGS. 1 through 4, including embodiments involving monitoring device 200, one of skill in the art will appreciate additional variations and details regarding the functionality of these embodiments that may be carried out by computing component 600. In this connection, it will also be appreciated by one of skill in the art upon studying the present disclosure that features and aspects of the various embodiments (e.g., systems) described herein may be implemented with respected to other embodiments (e.g., methods) described herein without departing from the spirit of the disclosure.

As used herein, the term component may describe a given unit of functionality that may be performed in accordance with one or more embodiments of the present application. As used herein, a component reference a module, and/or may be implemented utilizing any form of hardware, software, or a combination thereof. For example, one or more processors, controllers, ASICs, PLAs, PALs, CPLDs, FPGAs, logical components, software routines or other mechanisms may be implemented to make up a component. In embodiment, the various components described herein may be implemented as discrete components or the functions and features described may be shared in part or in total among one or more components. In other words, as would be apparent to one of ordinary skill in the art after reading this description, the various features and functionality described herein may be implemented in any given application and may be implemented in one or more separate or shared components in various combinations and permutations. Even though various features or elements of functionality may be individually described or claimed as separate components, one of ordinary skill in the art will understand upon studying the present disclosure that these features and functionality may be shared among one or more common software and hardware elements, and such description shall not require or imply that separate hardware or software components are used to implement such features or functionality.

Where components of the application are implemented in whole or in part using software, in one embodiment, these software elements can be implemented to operate with a computing or processing component capable of carrying out the functionality described with respect thereto. One such example computing component is shown in FIG. 6. Various embodiments are described in terms of this example-computing component 600. After reading this description, it will become apparent to a person skilled in the relevant art how to implement the application using other computing components or architectures.

Referring now to FIG. 6, computing component 600 may represent, for example, computing or processing capabilities found within a self-adjusting display, desktop, laptop, notebook, and tablet computers; hand-held computing devices (tablets, PDA's, smart phones, cell phones, palmtops, etc.); workstations or other devices with displays; servers; or any other type of special-purpose or general-purpose computing devices as may be desirable or appropriate for a given application or environment. Computing component 600 might also represent computing capabilities embedded within or otherwise available to a given device. For example, a computing component might be found in other electronic devices such as, for example navigation systems, portable computing devices, and other electronic devices that might include some form of processing capability.

Computing component 600 might include, for example, one or more processors, controllers, control components, or other processing devices, such as a processor 604. Processor 604 might be implemented using a general-purpose or special-purpose processing engine such as, for example, a microprocessor, controller, or other control logic. In the illustrated example, processor 604 is connected to a bus 602, although any communication medium can be used to facilitate interaction with other components of computing component 600 or to communicate externally.

Computing component 600 might also include one or more memory components, simply referred to herein as main memory 608. For example, preferably random access memory (RAM) or other static or dynamic memory, might be used for storing information and instructions to be executed by processor 604. Main memory 608 might also be used for storing temporary variables or other intermediate information during execution of instructions to be executed by processor 604. Computing component 600 might likewise include a read only memory (“ROM”) or other static storage device coupled to bus 602 for storing static information and instructions for processor 604.

The computing component 600 might also include one or more various forms of information storage mechanism 610, which might include, for example, a media drive 612 and a storage unit interface 620. The media drive 612 might include a drive or other mechanism to support fixed or removable storage media 614. For example, a hard disk drive, a solid state drive, a magnetic tape drive, an optical disk drive, a compact disc (CD) or digital video disc (DVD) drive (R or RW), or other removable or fixed media drive might be provided. Accordingly, storage media 614 might include, for example, a hard disk, an integrated circuit assembly, magnetic tape, cartridge, optical disk, a CD or DVD, or other fixed or removable medium that is read by, written to or accessed by media drive 612. As these examples illustrate, the storage media 614 can include a computer usable storage medium having stored therein computer software or data.

In alternative embodiments, information storage mechanism 610 might include other similar instrumentalities for allowing computer programs or other instructions or data to be loaded into computing component 600. Such instrumentalities might include, for example, a fixed or removable storage unit 622 and an interface 620. Examples of such storage units 622 and interfaces 620 can include a program cartridge and cartridge interface, a removable memory (for example, a flash memory or other removable memory component) and memory slot, a PCMCIA slot and card, and other fixed or removable storage units 622 and interfaces 620 that allow software and data to be transferred from the storage unit 622 to computing component 600.

Computing component 600 might also include a communications interface 624. Communications interface 624 might be used to allow software and data to be transferred between computing component 600 and external devices. Examples of communications interface 624 might include a modem or softmodem, a network interface (such as an Ethernet, network interface card, WiMedia, IEEE 802.XX or other interface), a communications port (such as for example, a USB port, IR port, RS232 port Bluetooth® interface, or other port), or other communications interface. Software and data transferred via communications interface 624 might typically be carried on signals, which can be electronic, electromagnetic (which includes optical) or other signals capable of being exchanged by a given communications interface 624. These signals might be provided to communications interface 624 via a channel 628. This channel 628 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to transitory or non-transitory media such as, for example, memory 608, storage unit 620, media 614, and channel 628. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the computing component 600 to perform features or functions of the present application as discussed herein.

Although described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the application, whether or not such embodiments are described and whether or not such features are presented as being a part of a described embodiment. Thus, the breadth and scope of the present application should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The use of the term “component” does not imply that the components or functionality described or claimed as part of the component are all configured in a common package. Indeed, any or all of the various components of a component, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

The details of some embodiments of the systems and methods of the present disclosure are set forth in this description and in some cases, in other portions of the disclosure. Other features, objects, and advantages of the disclosure will be apparent to one of skill in the art upon examination of the present disclosure, description, figures, examples, and claims. It is intended that all such additional systems, methods, devices, features, and advantages be included within this description (whether explicitly or by reference), be within the scope of the present disclosure, and be protected by one or more of the accompanying claims.

What is claimed is:

1. An incentive spirometry device, comprising:
a sensor measuring inspired air volume in an incentive spirometry device by tracking displacement of a piston within the incentive spirometer;
a processor configured to determine compliance by a patient using the incentive spirometer device based on a summation of counted successful breathing events over a period of time and to determine a reward based on the determined compliance;
a display presenting a visual indication indicating the reward.

2. The incentive spirometry device of claim 1, wherein the visual indication is at least one of: points, achievement badges, progress bars, virtual currency.

3. The incentive spirometry device of claim 1, wherein the visual indication is graphical visualization of a progress bar that is based on the determined compliance relative to the patient-specific incentive spirometry goal.

4. The incentive spirometry device of claim 1, further comprising:
an inertial measurement unit (IMU) sensor detecting movement of the incentive spirometer device,
wherein the processor is further configured to determine compliance based on a the movement detected by the IMU sensor.

5. The incentive spirometry device of claim 1, further comprising:
an inertial measurement unit (IMU) sensor detecting movement of the incentive spirometer device,
wherein the processor is further configured to supply of power to the user interface and the sensor based on the movement detected by the IMU sensor.

6. The incentive spirometry device of claim 1, wherein the sensor is integrated as part of an air chamber within which the piston is displaced.

7. The incentive spirometry device of claim 1, wherein the sensor is integrated as part of the piston.

8. The incentive spirometry device of claim 1, wherein the sensor is remote from the processor, and the sensor operatively connects to the processor over one of a wired or wireless connection.

9. The incentive spirometry device of claim 1, wherein the user interface, the sensor, the processor, and the display comprise a portable unit attachable to and detachable from an air chamber within which the piston is displaced.

10. The incentive spirometry device of claim 1, further comprising a counter to count successful inspiring air events.

11. The incentive spirometry device of claim 10, wherein the patient-specific incentive spirometry goal comprises a desired air volume of air inspired or exhaled by the patient.

12. The incentive spirometry device of claim 11, wherein the user interface comprises one or more switches enabling inputting of the desired volume of inspired air.

13. The incentive spirometry device of claim 12, wherein the one or more switches further enable bookmarking of at least one of an inspiring air event, a breathing event, and a patient state.

14. The incentive spirometry device of claim 13, further comprising a memory unit operatively connected to the processor, the memory unit storing the at least one of the inspiring air event, the breathing event, and the patient state.

15. The incentive spirometry device of claim 14, the memory unit further storing at least one of minimum and maximum inspired air volumes over the period of time or one or more subsets of the period of time.

16. A method, comprising:
executing an initialization function of a monitoring device in response to being operatively connected to an incentive spirometer, wherein the monitoring device comprises a sensor measuring inspired air volume in the incentive spirometer by tracking displacement of a piston within the incentive spirometer;
performing calibration to effectuate a default rest state of the piston;
configuring the monitoring device according to operating parameters of the monitoring device and the incentive spirometer; and
measuring and outputting inspiratory metrics with the monitoring device in response to use of the incentive spirometer.

17. The method of claim 16, wherein the measuring of the inspiratory metrics comprises receiving, by the monitoring device, a breathing event input, and determining satisfaction of an orientation position of the incentive spirometer relative to a preset orientation criterion.

18. The method of claim 16, further comprising re-performing the calibration in response to a determination that a position of the piston is not commensurate with the default rest state of the piston.

19. The method of claim 16, wherein the measuring and outputting of the inspiratory metrics comprises determining whether a dynamic inspiratory goal is met in accordance a user-specific configuration set forth by the monitoring device.

20. The method of claim 19, wherein the user-specific configuration comprises successive, different inspiratory goals.
